(19) United States
(12) Patent Application Publication
(10) Pub. No.: US 2018/0085174 A1
(43) Pub. Date: Mar. 29, 2018

(54) SUCTION DEVICE FOR USE IN IMAGE-GUIDED SINUS MEDICAL PROCEDURE

(71) Applicant: Acclarent, Inc., Irvine, CA (US)

(72) Inventors: Lauren Radtke, Irvine, CA (US); Mina W. Chow, Campbell, CA (US); Jephrey Rodriguez, Irvine, CA (US); Athanasios Papadakis, Newport Beach, CA (US); Ketan P. Muni, San Jose, CA (US); Frederick Kuhn, Savannah, GA (US); Ronan L. Jenkinson, Walnut Creek, CA (US); Jeffery J. Christian, Morgan Hill, CA (US); William Kane, Irvine, CA (US)

(21) Appl. No.: 15/358,251

(22) Filed: Nov. 22, 2016

Related U.S. Application Data
(60) Provisional application No. 62/398,786, filed on Sep. 23, 2016.

(51) Int. Cl. A61B 34/20; A61B 5/06; A61M 1/00; A61M 25/09; A61B 17/24

(52) U.S. Cl. CPC A61B 34/20; A61B 5/062; A61M 1/0023; A61B 2017/0046; A61B 17/24; A61B 2034/2051; A61M 2210/0681; A61M 25/09041

(57) ABSTRACT

An apparatus includes a navigational guidewire and a suction device. The navigational guidewire includes a sensing element, an outer member and a conductor. The sensing element is configured to respond to positioning within an electromagnetic field. The conductor extends along the length of the outer member and is in communication with the sensing element. The shaft assembly has an open distal end. The shaft assembly also defines a suction lumen and a guidewire lumen extending through the open distal end. The guidewire lumen is dimensioned to receive the sensing element of the navigational guidewire.

### PRIORITY

This application claims priority to U.S. Provisional Pat. App. No. 62/398,786, entitled “Suction Device for Use in Image-Guided Sinus Medical Procedure,” filed Sep. 23, 2016, the disclosure of which is incorporated by reference herein.

### BACKGROUND

In some instances, it may be desirable to operate within or adjacent to an anatomical passageway of a patient, such as performing an incision of mucosa, removal of bone, or dilation of an anatomical passageway. Such operations may occur within anatomical passageways such as ostia of paranasal sinuses (e.g., to treat sinusitis), the larynx, the Eustachian tube, or other passageways within the ear, nose, or throat, etc. In addition to the above described operations, or similar operations, it may be desirable to apply suction and/or irrigation within or adjacent to an anatomical passageway before, during, or after the above described operations, or similar operations. One method of applying suction within or adjacent to an anatomical passageway of a patient involves obtaining a suction device having an elongate shaft defining a lumen terminating at an open distal end of the elongated shaft, where the lumen is in fluid communication with an external suction source. An operator may then insert the distal end of the elongate shaft within the nostril or mouth of a patient toward a desired location within the patient. With the distal end of the elongate shaft inserted within the patient, an operator may manipulate the suction device and/or suction source in order to remove extraneous and/or undesired matter near or within an anatomical passageway of a patient. Applying suction and/or irrigation during an operation may be beneficial for multiple purposes as will be apparent to those skilled in the art.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.) so as to superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot, etc.) showing the real time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

Examples of electromagnetic IGS systems that may be used in ENT and sinus surgery include the InstaTrak ENT™ systems available from GE Medical Systems, Salt Lake City, Utah. Other examples of electromagnetic image guidance systems that may be modified for use in accordance with the present disclosure include but are not limited to the CARTO® 3 System by Biosense-Webster, Inc., of Diamond Bar, Calif.; systems available from Surgical Navigation Technologies, Inc., of Louisville, Colo.; and systems available from Calypso Medical Technologies, Inc., of Seattle, Wash.

When applied to functional endoscopic sinus surgery (FESS), balloon sinuplasty, and/or other ENT procedures, the use of image guidance systems allows the surgeon to achieve more precise movement and positioning of the surgical instruments than can be achieved by viewing through an endoscope alone. This is so because a typical endoscopic image is a spatially limited, 2 dimensional, line-of-sight view. The use of image guidance systems provides a real time, 3-dimensional view of all of the anatomy surrounding the operative field, not just that which is actually visible in the spatially limited, 2 dimensional, direct line-of-sight endoscopic view. As a result, image guidance systems may be particularly useful during performance of FESS, balloon sinuplasty, and/or other ENT procedures where a section and/or irrigation source may be desirable, especially in cases where normal anatomical landmarks are not present or are difficult to visualize endoscopically.

It may be desirable to provide features that further facilitate the use of an IGS navigation system and associated components in ENT procedures and other medical procedures. While several systems and methods have been made and used with respect to IGS and ENT surgery, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic view of an exemplary sinus surgery navigation system;

FIG. 2 depicts a perspective view of the head of a patient, with components of the navigation system of FIG. 1;

FIG. 3 depicts a side view of an exemplary hand-held suction instrument coupled with a vacuum assembly;

FIG. 4 depicts a perspective view of the hand-held suction instrument of FIG. 3;

FIG. 5 depicts a cross-sectional perspective view of the hand-held suction instrument of FIG. 3;

FIG. 6 depicts a cross-sectional side view of a guidewire manipulation assembly of the hand-held suction instrument of FIG. 3;

FIG. 7A depicts an elevational side view of the guidewire manipulation assembly of FIG. 6 in an unlocked position;

FIG. 7B depicts an elevational side view of the guidewire manipulation assembly of FIG. 6 in a locked position;

FIG. 8A depicts a cross-sectional front view of the guidewire manipulation assembly of FIG. 6 in the unlocked position, taken along line 8A-8A of FIG. 7A;

FIG. 8B depicts a cross-sectional front view of the guidewire manipulation assembly of FIG. 6 in the locked position, taken along line 8B-8B of FIG. 7B;

FIG. 9 depicts a cross-sectional side view of a shaft assembly of the hand-held suction instrument of FIG. 3;

FIG. 10 depicts a cross-sectional side view of the shaft assembly of FIG. 9;

FIG. 11 depicts an enlarged cross-sectional side view of the shaft assembly of FIG. 9, taken from circle 11 of FIG. 10;

FIG. 12 depicts a cross-sectional end view of the shaft assembly of FIG. 9, taken along line 12-12 of FIG. 11;

FIG. 13 depicts a cross-sectional end view of the shaft assembly of FIG. 9, taken along line 13-13 of FIG. 11;

FIG. 14 depicts a cross-sectional end view of the shaft assembly of FIG. 9, taken along line 14-14 of FIG. 11;

FIG. 15 depicts a cross-sectional front view of an exemplary alternative shaft assembly that may be readily incorporated into the hand-held suction instrument of FIG. 3;

FIG. 16 depicts a cross-sectional side view of another exemplary alternative shaft assembly that may be readily incorporated into the hand-held suction instrument of FIG. 3:

FIG. 17 depicts a cross-sectional end view of the shaft assembly of FIG. 16, taken along line 17-17 of FIG. 16;

FIG. 18 depicts a cross-sectional end view of the shaft assembly of FIG. 16, taken along line 18-18 of FIG. 16;

FIG. 19 depicts a perspective view of a distal end of another exemplary alternative shaft assembly that may be readily incorporated into the hand-held suction instrument of FIG. 3;

FIG. 20 depicts a cross-sectional side view of the shaft assembly of FIG. 19;

FIG. 21 depicts a side elevational view of an exemplary alternative hand-held suction instrument;

FIG. 22 depicts a cross-sectional side view of the hand-held suction instrument of FIG. 21;

FIG. 23 depicts a perspective view of another exemplary alternative hand-held suction instrument;

FIG. 24A depicts an elevational side view of the hand-held suction instrument of FIG. 23, where a guidewire manipulation assembly is in a retracted position;

FIG. 24B depicts a side elevational view of the hand-held suction instrument of FIG. 23, where the guidewire manipulation assembly of FIG. 24A is in an advanced position;

FIG. 25 depicts an exploded perspective view of the hand-held suction instrument of FIG. 23;

FIG. 26 depicts a perspective view of a handle assembly of the hand-held suction instrument of FIG. 23;

FIG. 27 depicts a cross-sectional side view of the handle assembly of FIG. 26;

FIG. 28 depicts an enlarged cross-sectional side view of the hand-held suction instrument of FIG. 23;

FIG. 29 depicts a perspective view of a removable shaft assembly of the hand-held suction instrument of FIG. 23;

FIG. 30 depicts another perspective view of a distal end of the removable shaft assembly of FIG. 29;

FIG. 31 depicts a cross-sectional side view of the removable shaft assembly of FIG. 29;

FIG. 32A depicts an enlarged cross-sectional side view, taken along line 32-32 of FIG. 23, of the removable shaft assembly of FIG. 29 being initially inserted into the handle assembly of FIG. 26;

FIG. 32B depicts an enlarged cross-sectional side view, taken along line 32-32 of FIG. 23, of the removable shaft assembly of FIG. 29 at an intermediate stage of insertion into the handle assembly of FIG. 26;

FIG. 32C depicts an enlarged cross-sectional side view, taken along line 32-32 of FIG. 23, of the removable shaft assembly of FIG. 29 fully inserted into and coupled with the handle assembly of FIG. 26;

FIG. 33 depicts a side elevational view of an exemplary pointer;

FIG. 34 depicts a cross-sectional side view of the pointer of FIG. 33;

FIG. 35 depicts a side elevational view of an exemplary Kerrison instrument with a guidewire lumen;

FIG. 36 depicts a side elevational view of an exemplary Freer elevator instrument with a guidewire lumen; and

FIG. 37 depicts a side elevational view of an exemplary sinus seeker instrument with a guidewire lumen.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Image Guided Surgery Navigation System

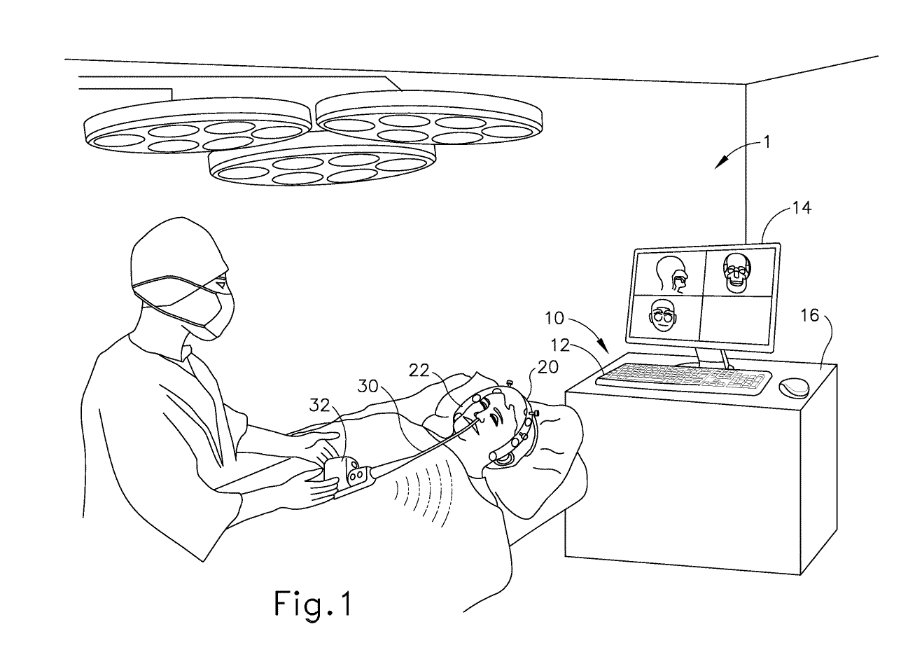

FIG. 1 shows an exemplary IGS navigation system (1) whereby an ENT procedure may be performed using IGS. In some instances, as will be described in greater detail below, IGS navigation system (1) is used during a procedure where suction adjacent to and/or within the procedure site is desired. However, it should be understood that IGS navigation system (1) may be readily used in various other kinds of procedures.

In addition to or in lieu of having the components and operability described herein IGS navigation system (1) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 8,190,389, entitled “Adapter for Attaching Electromagnetic Image Guidance Components to a Medical Device,” issued May 29, 2012, the disclosure of which is incorporated by reference herein; U.S. Pat. No. 8,123,722, entitled “Devices, Systems and Methods for Treating Disorders of the Ear, Nose and Throat,” issued Feb. 28, 2012, the disclosure of which is incorporated by reference herein; and U.S. Pat. No. 7,720,521, entitled “Methods and Devices for Performing Procedures within the Ear, Nose, Throat and Paranasal Sinuses,” issued May 18, 2010, the disclosure of which is incorporated by reference herein.

Similarly, in addition to or in lieu of having the components and operability described herein, IGS navigation system (1) may be constructed and operable in accordance with at least some of the teachings of U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pat. Pub. No. 2014/0200444, entitled “Guidewires for Performing Image Guided Procedures,” published Jul. 17, 2014, the disclosure of which is incorporated by reference herein; U.S. Pat. Pub. No. 2012/0245456, entitled “Adapter for Attaching Electromagnetic Image Guidance Components to a Medical Device,” published Sep. 27, 2012, the disclosure of which is incorporated by reference herein; U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein; U.S. Pat. Pub. No. 2008/0281156, entitled “Methods and Apparatus for Treating Disorders of the Ear Nose and Throat,” published Nov. 13, 2008, the disclosure of which is incorporated by reference herein; and U.S. Pat. Pub. No. 2007/0208252, entitled “Systems and Methods for Performing Image Guided Procedures within the Ear, Nose, Throat and Paranasal Sinuses,” published Sep. 6, 2007, the disclosure of which is incorporated by reference herein.

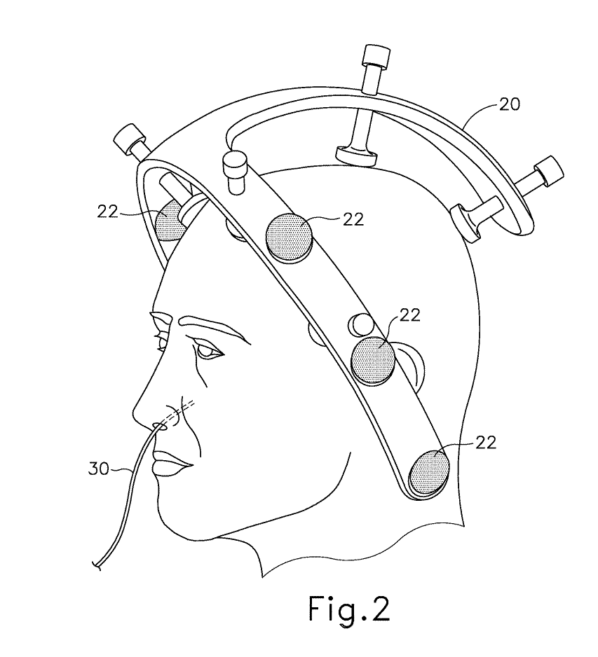

IGS navigation system (1) of the present example comprises a set of magnetic field generators (22). Before a surgical procedure begins, field generators (22) are fixed to the head of the patient. As best seen in FIG. 2, field generators (22) are incorporated into a frame (20), which is clamped to the head of the patient. While field generators (22) are secured to the head of the patient in this example, it should be understood that field generators (22) may instead be positioned at various other suitable locations and on various other suitable structures. By way of example only, field generators (22) may be mounted on an independent structure that is fixed to a table or chair on which the patient is positioned, on a floor-mounted stand that has been locked in position relative to the head of the patient, and/or at any other suitable location(s) and/or on any other suitable structure(s).

Field generators (22) are operable to generate an electromagnetic field around the head of the patient. In particular, field generators (22) are operated so as to transmit alternating magnetic fields of different frequencies into a region in proximity to frame (20). Field generators (22) thereby enable tracking of the position of a navigation guidewire (30) that is inserted into a nasal sinus of the patient and in other locations within the patient's head. Various suitable components that may be used to form and drive field generators (22) will be apparent to those of ordinary skill in the art in view of the teachings herein.

IGS navigation system (1) of the present example further comprises a processor (10), which controls field generators (22) and other elements of IGS navigation system (1). Processor (10) comprises a processing unit communicating with one or more memories. Processor (10) of the present example is mounted in a console (16), which comprises operating controls (12) that include a keypad and/or a pointing device such as a mouse or trackball. A physician uses operating controls (12) to interact with processor (10) while performing the surgical procedure.

Console (16) also connects to other elements of system (1). For instance, as shown in FIG. 1 a coupling unit (32) is secured to the proximal end of navigation guidewire (30). Coupling unit (32) of this example is configured to provide wireless communication of data and other signals between console (16) and navigation guidewire (30). In some versions, coupling unit (32) simply communicates data or other signals from navigation guidewire (30) to console (16) uni-directionally, without also communicating data or other signals from console (16). In some other versions, coupling unit (32) provides bidirectional communication of data or other signals between navigation guidewire (30) to console (16). While coupling unit (32) of the present example couples with console (16) wirelessly, some other versions may provide wired coupling between coupling unit (32) and console (16). Various other suitable features and functionality that may be incorporated into coupling unit (32) will be apparent to those of ordinary skill in the art in view of the teachings herein.

Processor (10) uses software stored in a memory of processor (10) to calibrate and operate system (1). Such operation includes driving field generators (22), processing data from navigational guidewire (30), processing data from operating controls (12), and driving display screen (14). The software may be downloaded to processor (10) in electronic form, over a network, for example, or it may, alternatively or additionally, be provided and/or stored on non-transitory tangible media, such as magnetic, optical, or electronic memory.

Processor (10) is further operable to provide video in real time via display screen (14), showing the position of the distal end of navigational guidewire (30) in relation to a video camera image of the patient's head, a CT scan image of the patient's head, and/or a computer generated three-dimensional model of the anatomy within and adjacent to the patient's nasal cavity. Display screen (14) may display such images simultaneously and/or superimposed on each other. Moreover, display screen (14) may display such images during the surgical procedure. Such displayed images may also include graphical representations of instruments that are inserted in the patient's head, such as navigational guidewire (30), such that the operator may view the virtual rendering of the instrument at its actual location in real time. Such graphical representations may actually look like the instrument or may be a much simpler representation such as a dot, crosshairs, etc. By way of example only, display screen (14) may provide images in accordance with at least some of the teachings of U.S. Pub. No. 2016/0008083, entitled “Guidewire Navigation for Sinuplasty,” published Jan. 14, 2016, the disclosure of which is incorporated by reference herein. In the event that the operator is also using an endoscope, the endoscopic image may also be provided on display screen (14). The images provided through display screen (14) may help guide the operator in maneuvering and otherwise manipulating instruments within the patient's head.

In the present example, navigational guidewire (30) includes one or more coils at the distal end of navigational guidewire (30). When such a coil is positioned within an electromagnetic field generated by field generators (22), movement of the coil within that magnetic field may generate electrical current in the coil, and this electrical current may be communicated along the electrical conduit(s) in navigational guidewire (30) and further to processor (10) via coupling unit (32). This phenomenon may enable IGS navigation system (00) to determine the location of the distal end of navigational guidewire (30) within a three-dimensional space as will be described in greater detail below. In particular, processor (10) executes an algorithm to calculate location coordinates of the distal end of navigational guidewire (30) from the position related signals of the coil(s) in navigational guidewire (30). While a coil is incorporated into navigational guidewire (30) to provide a navigational sensor in the present example, it should be understood that any other suitable components may be incorporated into navigational guidewire (30) to provide a navigational sensor.

In some instances, navigational guidewire (30) is used to generate a three-dimensional model of the anatomy within and adjacent to the patient's nasal cavity; in addition to being used to provide navigation for dilation catheter system (1) within the patient's nasal cavity. Alternatively, any other suitable device may be used to generate a three-dimensional model of the anatomy within and adjacent to the patient's nasal cavity before navigational guidewire (30) is used to provide navigation for dilation catheter system (1) within the patient's nasal cavity. By way of example only, a model of this anatomy may be generated in accordance with at least some of the teachings of U.S. Pub. No. 2016/0310042, entitled “System and Method to Map Structures of Nasal Cavity,” published Oct. 27, 2016, the disclosure of which is incorporated by reference herein. Still other suitable ways in which a three-dimensional model of the anatomy within and adjacent to the patient's nasal cavity may be generated will be apparent to those of ordinary skill in the art in view of the teachings herein. It should also be understood that, regardless of how or where the three-dimensional model of the anatomy within and adjacent to the patient's nasal cavity is generated, the model may be stored on console (16). Console (16) may thus render images of at least a portion of the model via display screen (14) and further render real-time video images of the position of navigational guidewire (30) in relation to the model via display screen (14).

II. Exemplary Suction Device for Image Guided Surgical Procedure

In some instances, it may be useful to use a suction instrument while also utilizing IGS navigation system (1). Utilizing a IGS navigation system (1) in conjunction with a suction instrument may allow an operator to provide better placement of the suction instrument within the patient. In addition to providing navigation capabilities to a suction instrument, guidewire (30) of IGS navigation system (1) may be utilized by an operator in conjunction with the suction instrument in order to probe around mucosa of a patient without causing severe damage.

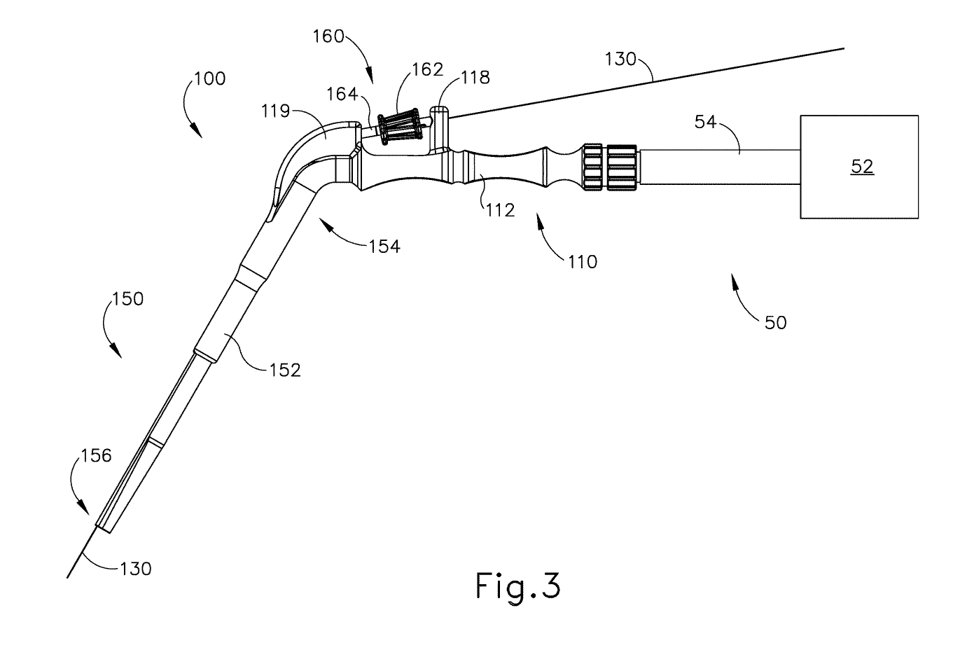

FIG. 3 shows an exemplary hand-held suction instrument (100) coupled with an exemplary vacuum system (50). Vacuum system (50) includes a suction source assembly (52) and a connecting tube (54). Connecting tube (54) provides fluid communication between suction source assembly (52) and a designated suction lumen (125) of exemplary hand-held suction instrument (100). Suction source assembly (52) is configured to generate enough suction to pull unwanted fluid/matter through designated suction lumen (125) and connecting tube (54) toward a reservoir and/or exit port of suction source assembly (52). Suction source assembly (52) may have any number of suitable components to provide suitable suction, storage, and disposal of fluid/matter traveling through suction lumen (125) and connecting tube (54) as would be apparent to one having ordinary skill in the art in view of the teachings herein.

While the current example above and examples described below envision suction instruments, it should be understood that suction lumens described below could be used alternatively as irrigation lumens, or any one suitable type of fluid transfer as would be apparent to one having ordinary skill in the art in view of the teachings herein.

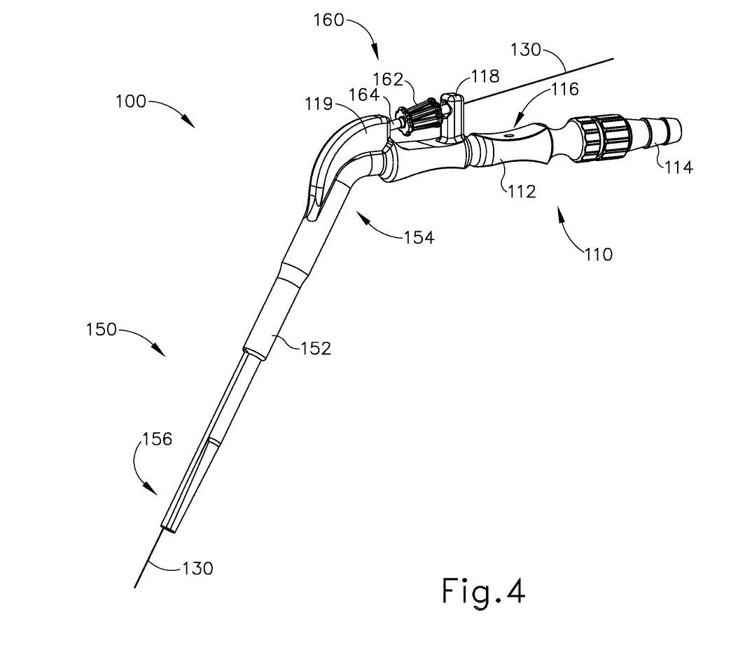

As shown in FIGS. 3-5, hand-held suction instrument (100) includes a handle assembly (110), an elongate shaft assembly (150) extending distally and obliquely from handle assembly (110), a guidewire manipulation assembly (160) rotatably connected to handle assembly (110). A guidewire (130) extends through a guidewire manipulation assembly (160), elongate shaft assembly (150), and a portion of handle assembly (112). It should be understood that guidewire (130) is substantially similar to guidewire (30) described above, including the incorporation of a navigation coil at the distal end of guidewire (130). Therefore, a distal end of guidewire (130) may be placed within a patient and located utilizing IGS navigation system (1). Additionally, a proximal end of guidewire (130) may be connected to coupling unit (32) such that guidewire (130) may communication with console (16).

As will be described in greater detail below, guidewire manipulation assembly (160) is configured to slidably receive guidewire (130), then selectively lock guidewire (130) relative to guidewire manipulation assembly (160), then rotate guidewire (130) about its own longitudinal axis relative to the rest of hand-held suction instrument (100).

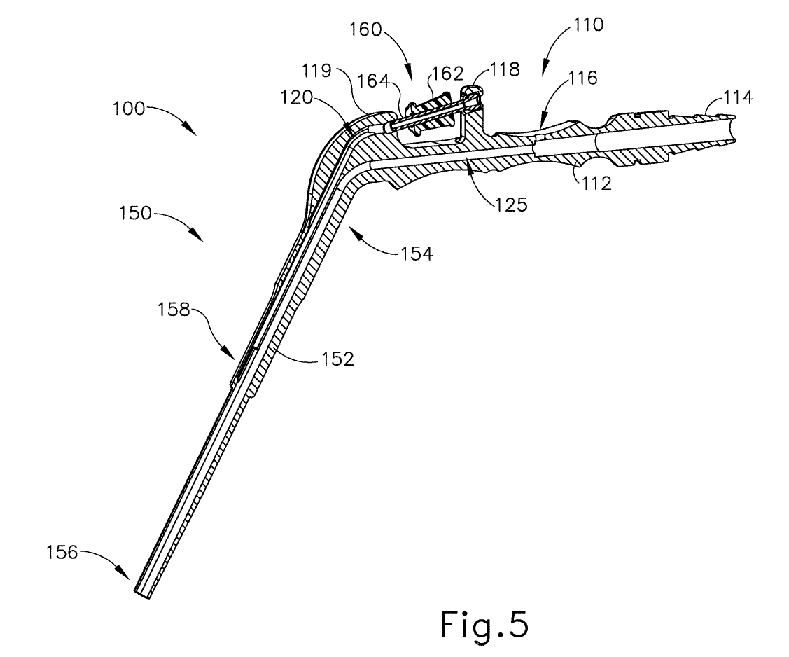

As best seen in FIG. 5, hand-held suction instrument (100) defines a guidewire lumen (120) and suction lumen (125), both of which terminate at an open distal end (156) of shaft assembly (150). It should be understood that FIG. 5 omits guidewire (130) for purposes of clarity. As will be described in greater detail below, guidewire lumen (120) may slidably and rotatably house a portion guidewire (130) such that guidewire (130) may rotate about its own longitudinal axis and translate through open distal end (156) of shaft assembly (150). Suction lumen (125) extends from a barbed connection (114) through handle assembly (110) and elongate shaft assembly (150) to provide fluid communication between barbed connection (114) and open distal end (156). Open distal end (156) and a portion of shaft assembly (150) are dimensioned to be inserted into an anatomical passageway of a patient via a nostril. For instance, shaft assembly (150) may be inserted through a patient's nostril to position distal end (156) in the patient's nasal cavity. However, it should be understood open distal end (156) of shaft assembly (150), and the rest of shaft assembly (150), may be dimensioned to be inserted in a variety of anatomical passageways as would be apparent to one having ordinary skill in the art in view of the teachings herein.

Handle assembly (110) includes a body (112) defining a suction control port (116), proximal barbed connector (114), and a pair of mounts (118, 119). Barbed connector (114) is dimensioned to couple with connecting tube (54) in order to provide fluid communication between suction lumen (125) and connecting tube (54). While in the current example, a barbed connector (114) is utilized to provide fluid communication between connecting tube (54) and suction lumen (125) of hand-held suction instrument (100), any other suitable connector may be utilized as would be apparent to one having ordinary skill in the art in view of the teachings herein.

Body (112) of handle assembly (110) is dimensioned so that an operator may grip handle assembly (110) and control guidewire manipulation assembly (160) with one hand. This may allow for an operator to control hand-held suction instrument (100) with one hand while controlling an endoscope or other instrument with another hand. Therefore, an operator may be able to utilize hand-held suction instrument (100) in conjunction with visuals provided by both IGS navigation system (1) and an endoscope. Alternatively, an operator may utilize hand-held suction instrument (100) with just an endoscope or just IGS navigation system (1). By way of example only, in settings where hand-held suction instrument (100) is used with an endoscope, such an endoscope may be constructed and operable in accordance with at least some of the teachings of U.S. Pub. No. 2010/0030031, the disclosure of which is incorporated by reference herein. Similarly, such an endoscope may be configured and operable like the Acclarent Cyclops™ Multi-Angle Endoscope by Acclarent, Inc. of Menlo Park, Calif. Other suitable forms that an endoscope may take will be apparent to those of ordinary skill in the art in view of the teachings herein.

Suction port (116) is placed on handle assembly (110) such that an operator may selectively cover suction port (116) with the same hand grasping body (112) during use. Suction port (116) connects the exterior of body (112) with suction lumen (125). Suction port (116) is dimensioned to act as a vent for suction lumen (125) when vacuum assembly (50) is connected and providing suction through suction lumen (125). Therefore, when suction source assembly (52) is activated and providing suction through connecting tube (54) and suction lumen (125), an operator may prevent suction from occurring at open distal end (156) of shaft assembly (150) by leaving suction port (116) open. In other words, suction source assembly (52) will simply draw in atmospheric air via suction port (116) when suction port (116) is open. If an operator desires to provide suction at open distal end (156) of shaft assembly (150), the operator may cover suction port (116). In other words, suction port (116) allows the operator to selectively activate suction at open distal end (156) of shaft assembly (150) even if vacuum assembly (50) is continuously activated and properly connected to hand-held suction instrument (100).

As will be described in greater detail below, mounts (118, 119) rotatably support guidewire manipulation assembly (160) such that guidewire manipulation assembly (160) may rotate guidewire (130) about the longitudinal axis defined by guidewire (130).

Shaft assembly (150) includes a shaft body (152) extending from a bent proximal portion (154) to open distal end (156). As described above, and as will be described in greater detail below, shaft body (152) defines portions of guidewire lumen (120) and suction lumen (125) such that guidewire (130) may translate through open distal end (156) and suction lumen (125) may carry unwanted fluid/matter away from open distal end (156). Bent proximal portion (154) bends the rest of elongate shaft assembly (150) away from the longitudinal axis of handle assembly (110), such that the distal portion of shaft assembly (150) defines an oblique angle with the longitudinal axis of handle assembly (110). Bent proximal portion (154) may form any suitable number of angles with the longitudinal axis of handle assembly (110) as would be apparent to one having ordinary skill in the art in view of the teachings herein. In the current example, open distal end (156) is parallel with the longitudinal axis defined by shaft body (152), however this is merely optional. Open distal end (156) may form any suitable number of curves and/or bends relative to the longitudinal axis defined by shaft body (152) as would be apparent to one having ordinary skill in the art in view of the teachings herein.

As will be described in greater detail below, elongate shaft assembly (150) also includes a transitional off-set portion (158) located between bent proximal portion (154) and open distal end (156). Transitional off-set portion (158) may be dimensioned to provide a desired amount of frictional contact between the interior of guidewire lumen (120) and guidewire (130) such that guidewire (130) does not accidentally move relative to hand-held suction instrument (100). In other words, transitional off-set portion (158) may increase the minimal force required to act on guidewire (130) in order to move guidewire (130) relative to hand-held suction instrument (100).

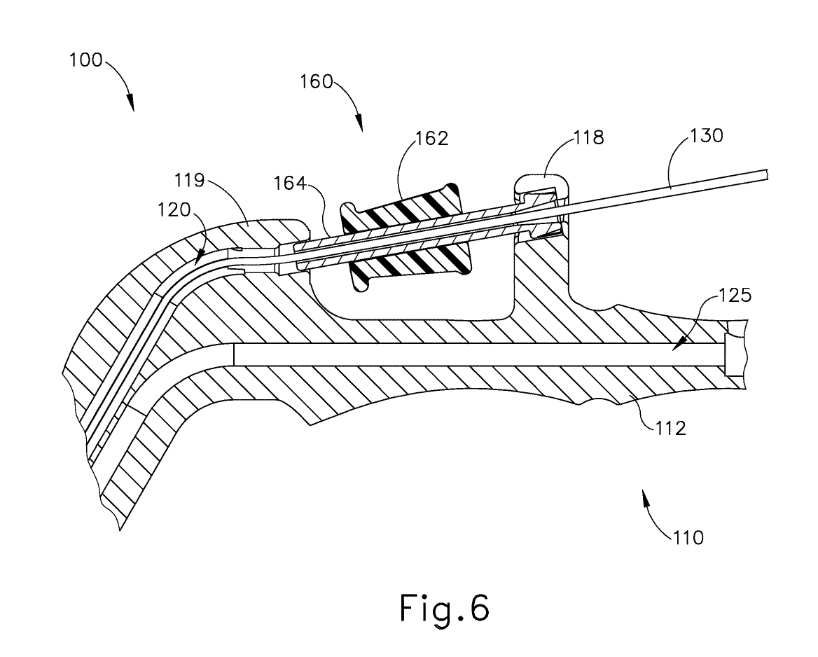

As best shown in FIG. 6, guidewire manipulation assembly (160) includes a locking grip (162) and a collet (164). Locking grip (162) includes a camming surface (161) that defines an interior channel (163). Collet (164) is inserted through interior channel (163) of locking grip (162) to slidably couple locking grip (162) to collet (164). As will be described in greater detail below, locking grip (162) may translate along collet (164) in order to selectively lock guidewire (130) relative to guidewire manipulation assembly (160). Additionally, locking grip (162) may be rotated while collet (164) is locked with guidewire (130) in order to rotate collet (164) and guidewire (130) about the longitudinal axis defined by guidewire (130).

Collet (164) extends between mounts (118, 119) of handle assembly (110). Collet (164) may rotate about its own longitudinal axis relative to mounts (118, 119). Mounts (118, 119) and collet (164) define a portion of guidewire lumen (120). Therefore, as shown in FIG. 6, a distal end of guidewire (130) may be inserted through mount (118), collet (164), and then mount (119) via guidewire lumen (120). Collet (164) defines a pair of slots (168). Collet (164) may be formed of a resilient material such that the portion of collet (164) defining slots (168) may flex toward and away from each other to a first position in response to external forces; and also flex back to a rested position in response to removal of external forces. Additionally, collet (164) includes a pair of protrusions (166) extending along the portion of collet (164) defining slots (168).

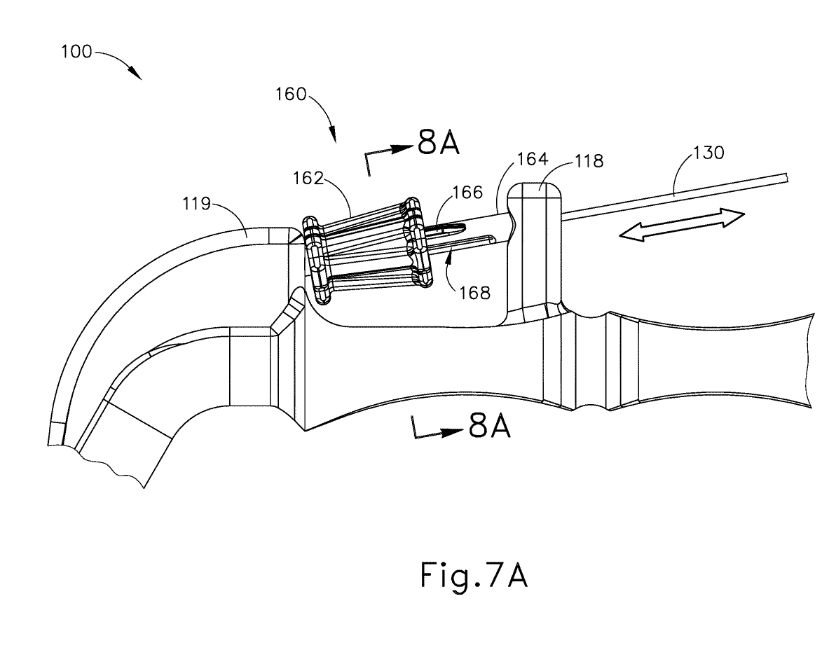

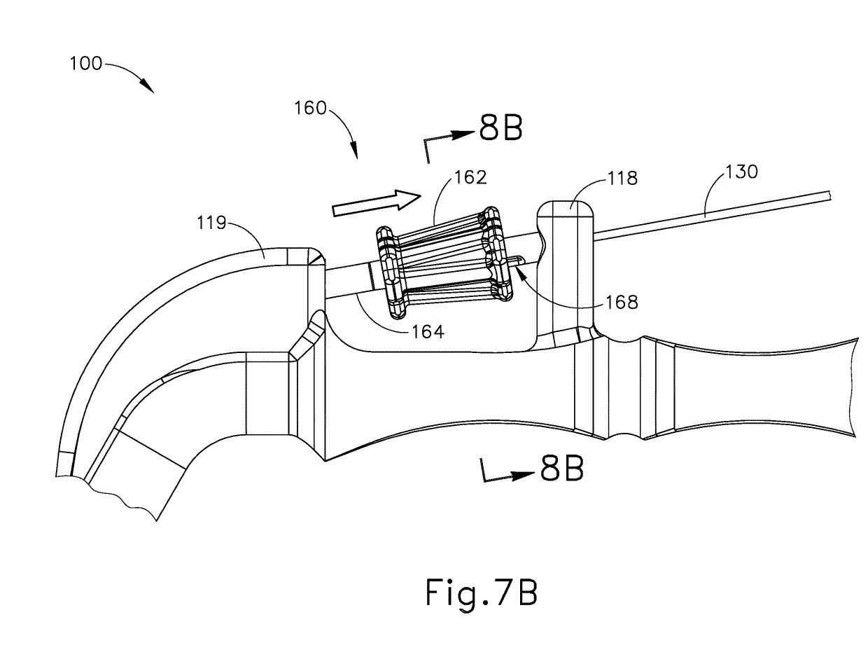

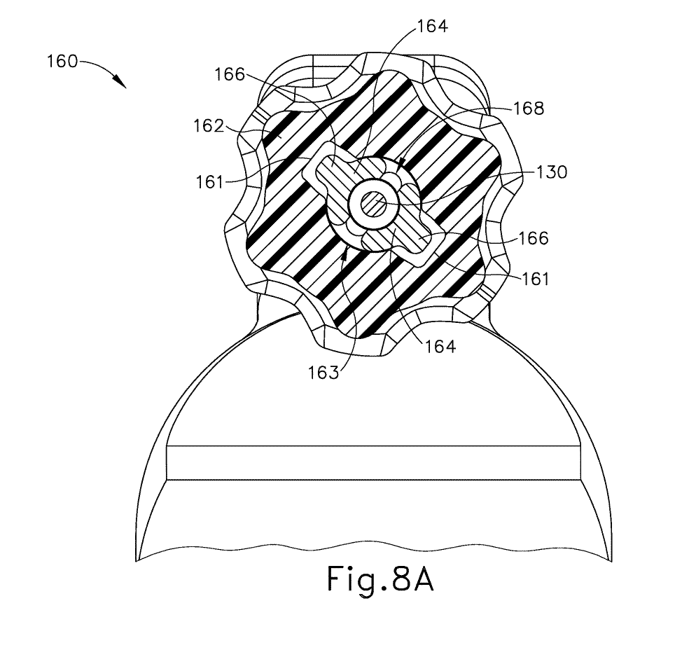

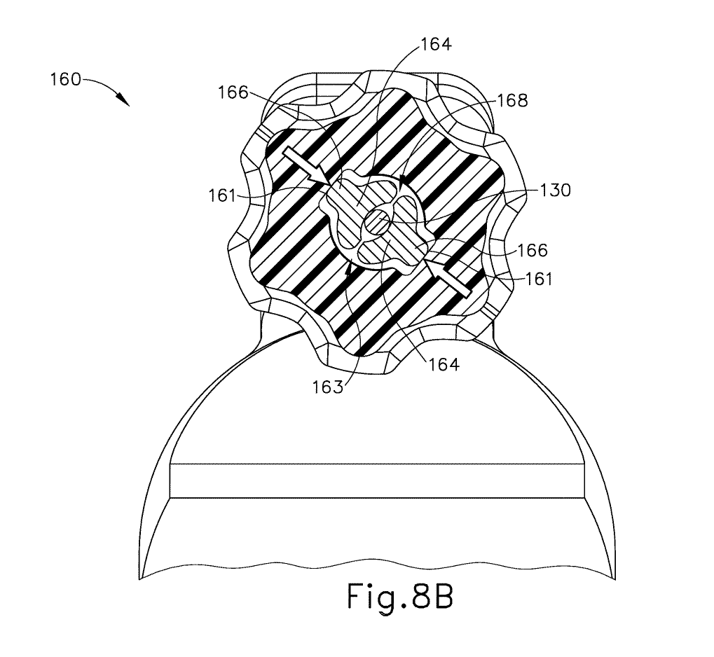

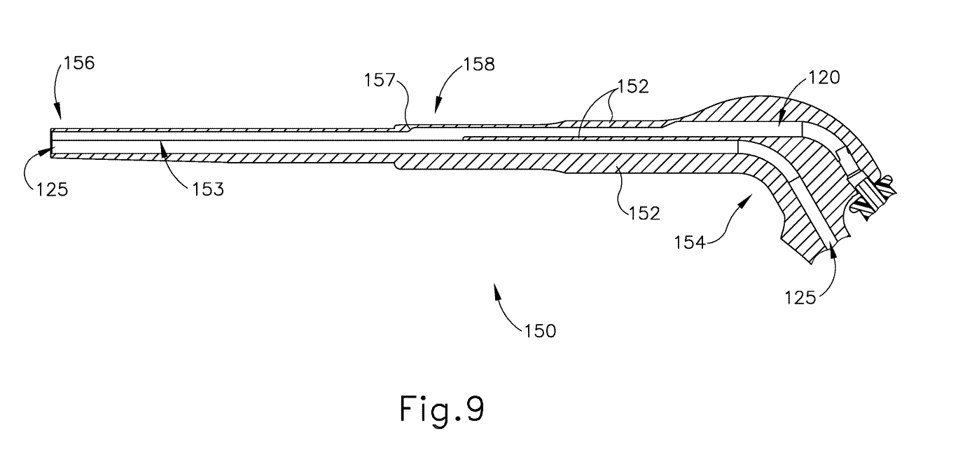

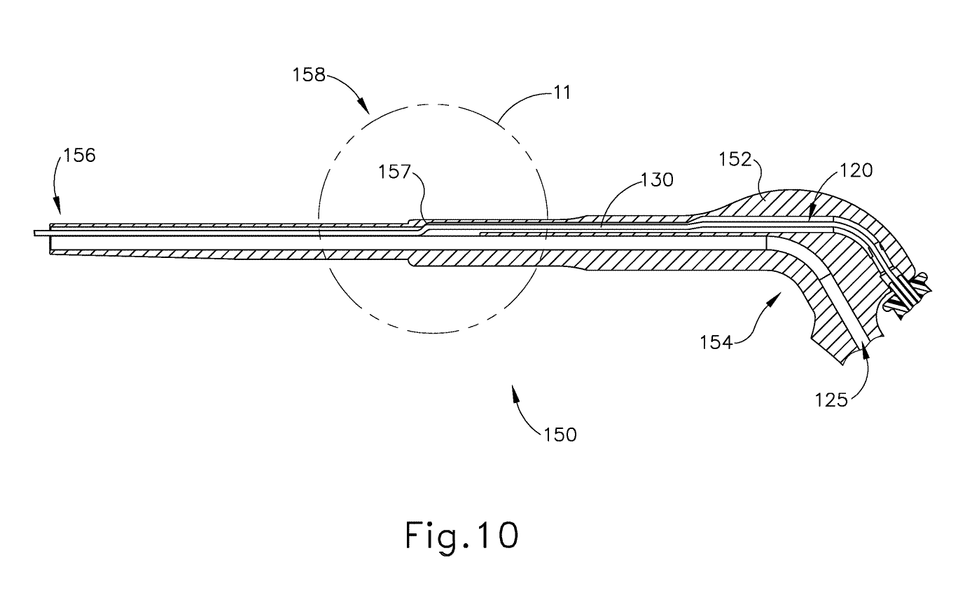

FIGS. 7A-8B show guidewire manipulation assembly (160) coupling with guidewire (130). Locking grip (162) may translate along collet (164) from an unlocked position (shown in FIGS. 7A and 8A) to a locked position (shown in FIGS. 7B and 8B). As best shown in FIG. 7A, when locking grip (162) is in the unlocked position, guidewire (130) may translate within guidewire lumen (120) distally or proximally. Therefore, when locking grip (162) is in the unlocked position, an operator may position guidewire (130) to a desired location relative to hand-held suction instrument (100). An operator may move guidewire (130) relative to hand-held suction instrument (100) for navigation purposes, for probing around mucosa, or any other suitable purpose as would be apparent to one having ordinary skill in the art in view of the teachings herein.

As best shown in FIG. 8A, camming surface (162) of locking grip (162) is positioned to not make contact with protrusions (166) of collet (164). Therefore, locking grip (162) does not impart any external forces on collet (164) to flex collet (164) against guidewire (130). When locking grip (162) translates along collet (164) toward the locked position, the dimensions of interior channel (163) and/or protrusions (166) change to encourage contact between cam surface (161) and protrusions (166). When in the locked position, as best shown in FIG. 8B, camming surface (162) contacts protrusions (166) to deflect collet (164) radially inwardly to narrow the dimension of slots (168). Collet (164) thus bears against guidewire (130) to provide friction that effectively locks guidewire (130) in place relative to collet (164). When collet (164) and guidewire (130) are thus locked together, rotation of locking grip (162) around the longitudinal axis of collet (164) also unitarily rotates guidewire (130) about the longitudinal axis of guidewire (130).

In the present example, locking grip (162) is configured to snap fit with collet (164) when translated into the locking position, such that locking grip (162) remains in the locking position unless an operator forces locking grip (162) toward the unlocked position with sufficient force. Due to the resilient nature of collet (164), once locking grip (162) translates from the locked position (shown in FIGS. 7B and 8B) to the unlocked position (shown in FIGS. 7A and 8A), portions of collet (164) defining slots (168) expand away from each other, increasing the dimension of slot (168) and allowing translation of guidewire (130) relative to guidewire manipulation assembly (160).

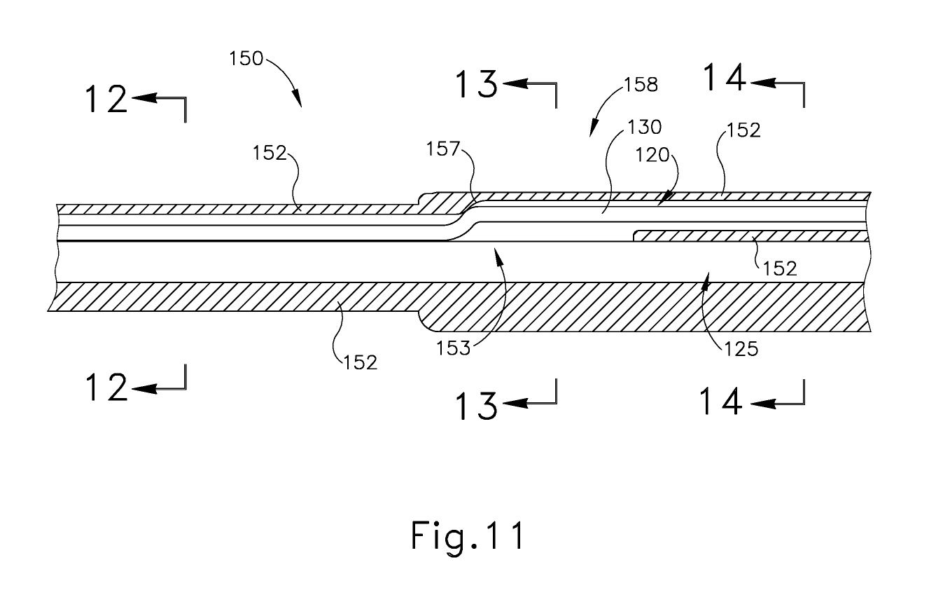

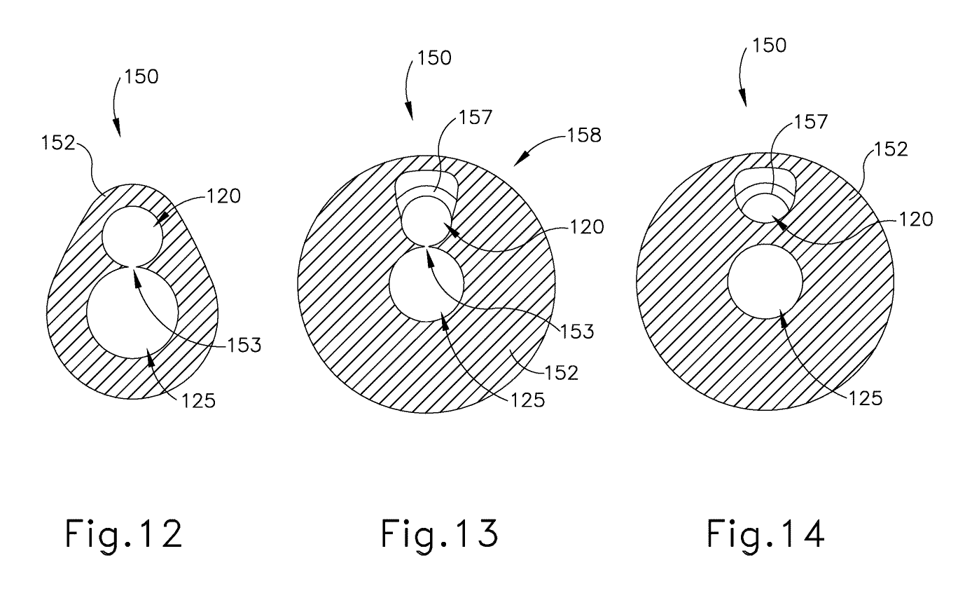

As described above, and will be described in greater detail below, shaft assembly (150) includes transitional off-set portion (158) that may be dimensioned to provide a desired amount of frictional contact between the interior of guidewire lumen (120) and guidewire (130) such that guidewire (130) does not accidentally move relative to hand-held suction instrument (100). As best seen in FIGS. 11-14, a portion of shaft assembly (150) proximal to transitional off-set portion (158) separates guidewire lumen (120) and suction lumen (125) with shaft body (152); while guidewire lumen (120) and suction lumen (125) are in fluid communication with each other via a gap (153) along shaft assembly (150) transitional off-set portion (158). It should be understood that guidewire (130) is purposely omitted from FIGS. 12-14 for purposes of clarity. Gap (153) is dimensioned to provide clearance for suction lumen to not be blocked by guidewire (130) during suction, thereby enabling suction flow even while guidewire (130) is positioned in guidewire lumen (120). It should also be understood that gap (153) is small enough to block guidewire (130) from transitioning from guidewire lumen (120) to suction lumen (125), which may increase guidewire (130) integrity during use. Fluid communication between guidewire lumen (120) and suction lumen (125) via gap (153) may allow suction within suction lumen (125) to drag guidewire (130) toward gap (153), therefore increasing the friction between interior of guidewire lumen (120) and guidewire (130). It should be understood that gap (153) of the current example is merely optional, and guidewire lumen (120) and suction lumen (125) may be fluidly isolated from each other along shaft assembly (150) to open distal end (156).

As best seen in FIGS. 11 and 13, transitional off-set portion (158) includes a slanted wall (157). Slanted wall (157) is configured to contact guidewire (130) in order to force guidewire (130) downwardly when extending from off-set portion (158) toward open distal end (156). This contact may also increase friction between guidewire (130) and various portions of guidewire lumen (120). The amount of friction may be varied based on the structural configuration of transitional off-set portion (168), including the height, length, and width of slanted wall (157); the distance between slanted wall (157) and the longitudinal transition between where shaft body (152) separates lumens (120, 125) and gap connects lumens (120, 125); and/or any other structural configuration aspects that would be apparent to one having ordinary skill in the art in view of the teachings herein. While the current example shows shaft assembly (150) having a transitional off-set portion (158), it should be understood that transitional off-set portion (158) is merely optional, and may be omitted entirely.

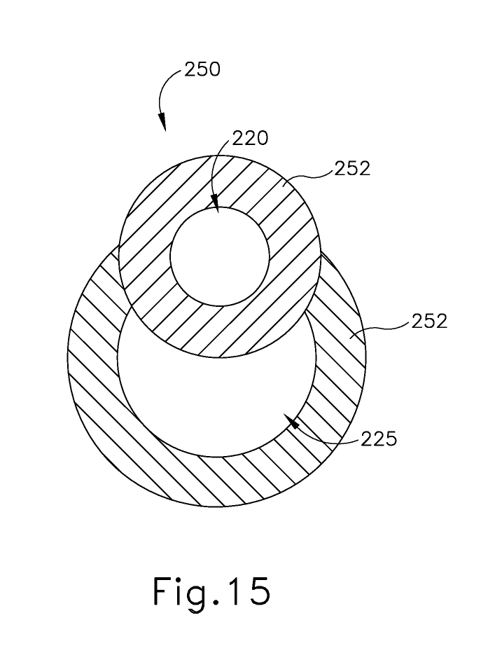

FIG. 15 shows an alternative shaft assembly (250) that may be readily incorporated into hand-held suction instrument (100) in replacement of shaft assembly (150) described above. It should be understood guidewire (130) is purposely omitted for clarity. Shaft assembly (250) of this example includes an alternative guidewire lumen (220) and an alternative suction lumen (225), which may be substantially similar to guidewire lumen (120) and suction lumen (225) described above respectively, with differences described below. In particular, a portion of body (252) defining both guidewire lumen (220) and suction lumen (225) is “saddled” within suction lumen (225) as to modify the cross-sectional area of the portion of suction lumen (225) physically separated from guidewire lumen (220). This arrangement may increase structural integrity of shaft assembly (250) and may also decrease cross-sectional area of shaft assembly (250).

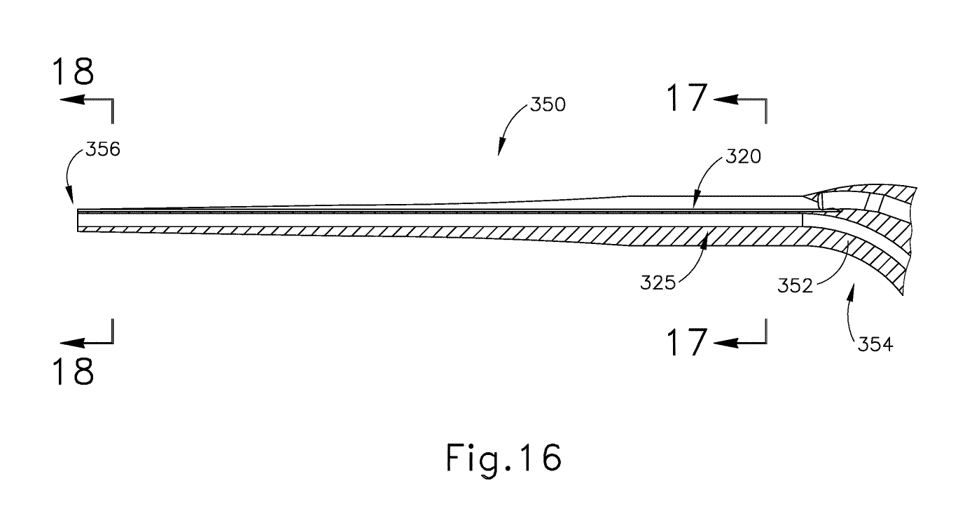

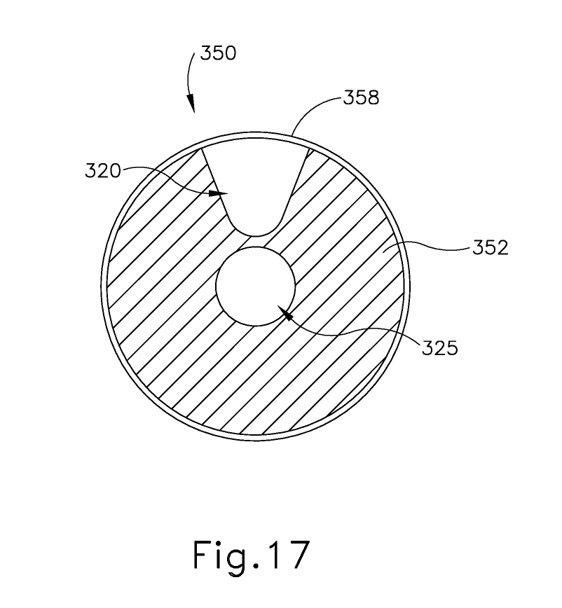

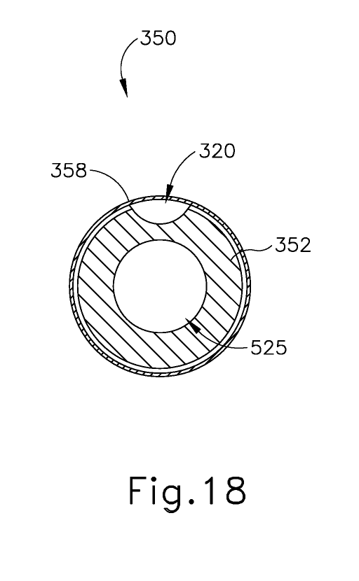

FIGS. 16-18 show another alternative shaft assembly (350) that may be readily incorporated into hand-held suction instrument (100) in replacement of shaft assembly (150) described above. It should be understood that guidewire (130) is purposely omitted for clarity. Shaft assembly (350) includes a shaft body (352) extending from a bent proximal portion (354) to an open distal end (356). Shaft body (352), bent proximal portion (354), and open distal end (356) may be substantially similar to shaft body (152), bent proximal portion (154), and open distal end (156) described above, respectively, with differences described below.

In the example shown in FIGS. 16-18, shaft body (352) defines a suction lumen (325) and a portion of a guidewire lumen (320), which may be substantially similar to suction lumen (125) and guidewire lumen (120) described above, respectively, with differences described below. Shaft assembly (350) also includes a heat shrink cover (358) that extends around body (352) from bent proximal portion (354) all the way toward open distal end (356). The portion ofguidewire lumen (320) defined by shaft body (352) is in the shape of “U,” while the cross-sectional area of guidewire lumen (320) is closed in by a portion of heat shrink cover (358) encompassing shaft body (352). In the current example, guidewire lumen (320) is physically separated from suction lumen (325) such that the two are not in fluid communication with each other along the entire length of shaft assembly (350) up to open distal end (356), which may accommodate more suction through suction lumen (325) while maintaining guidewire (130) integrity. As body (352) of shaft assembly (350) narrows toward open distal end (358), so does the “U” shape cross-section of guidewire lumen (320). While the current example utilizes a “U” shape cross-section for guidewire lumen (320), any other suitable shape may be used, such as a “W,” a “3,” a “a V,” etc.

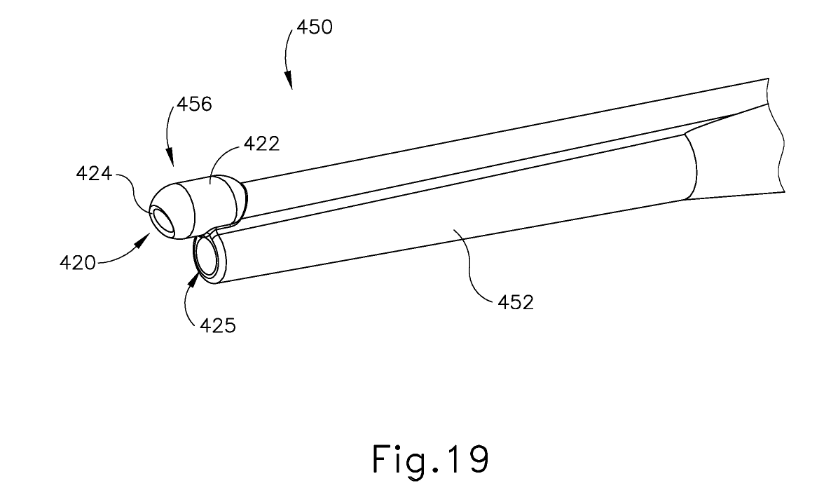

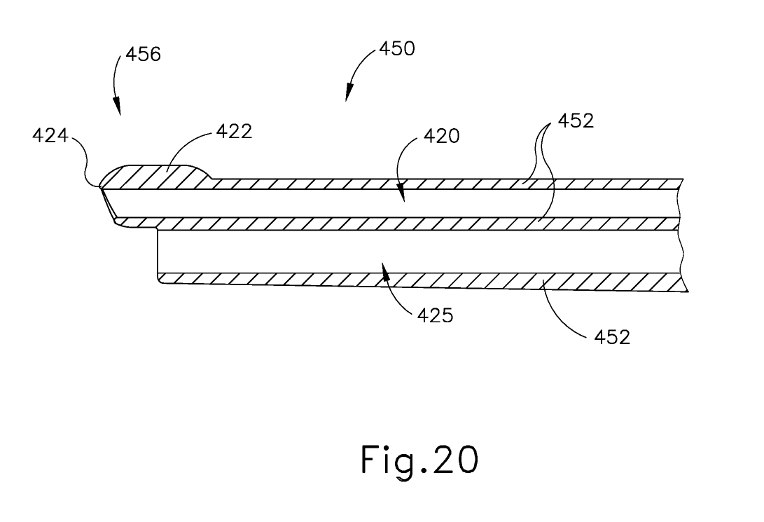

FIGS. 19-20 show another alternative shaft assembly (450) that may be readily incorporated into hand-held suction instrument (100) described above, in replacement of shaft assembly (150) described above. It should be understood that guidewire (130) is purposely omitted for clarity. It should be understood that shaft assembly (450) is substantially similar to shaft assembly (150) described above, with differences described below. Shaft assembly (450) includes a shaft body (452) extending toward an open distal end (456), which are substantially similar to shaft body (152) and open distal end (156) described above, respectively, with differences described below. Shaft body (452) also defines a guidewire lumen (420) and a suction lumen (425), which are substantially similar to guidewire lumen (120) and suction lumen (125) described above, respectively, with differences described below. In particular, guidewire lumen (420) terminates distally relative to the termination of suction lumen (425). Additionally, guidewire lumen (420) terminates into a ball tip (422) having a slanted open distal face (424). Ball tip (422) and slanted open distal face (424) may allow an operator to atraumatically probe and elevate or otherwise move anatomical structures via ball tip (422) at open distal end (456) of shaft assembly (450).

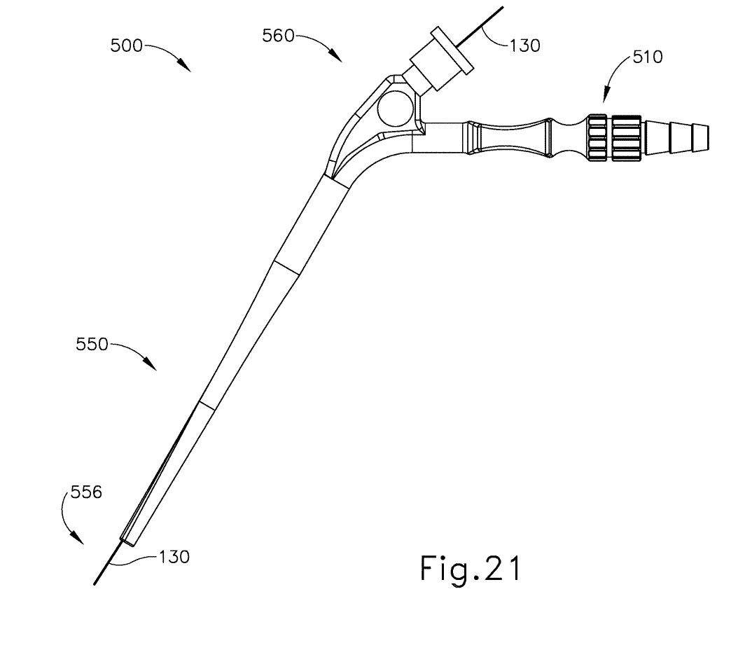

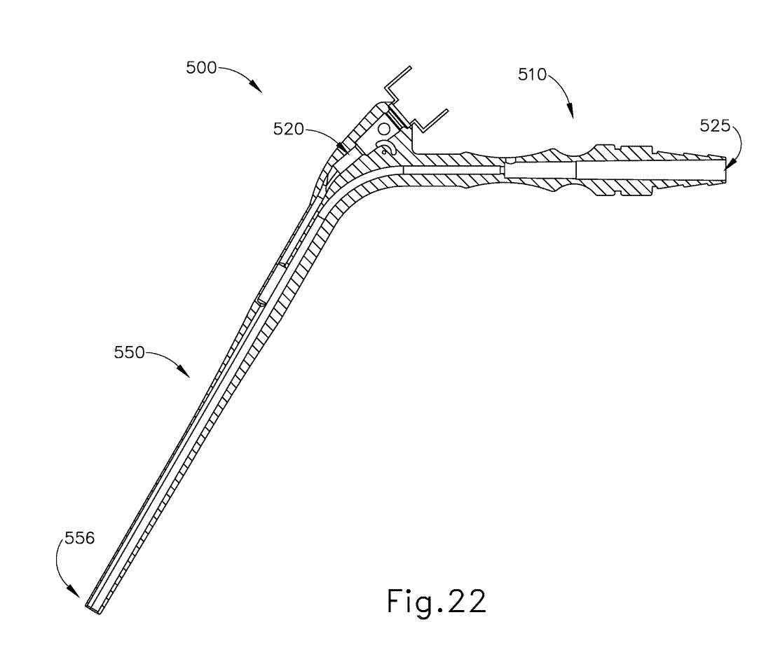

FIGS. 21-22 show an alternative hand-held suction instrument (500) that may be used in place of hand-held suction instrument (100) described above. Hand-held suction instrument (500) includes a handle assembly (510) and a shaft assembly (550) that are substantially similar to handle assembly (110) and shaft assembly described above. Therefore, a guidewire lumen (520) extends through shaft assembly (550) and a suction lumen (525) extends through handle assembly (510) and shaft assembly (550). However, instead of guidewire manipulation assembly (160), hand-held suction instrument (500) includes a rotatable guidewire acceptor (560), which is rotatably coupled to handle assembly (550). As best seen in FIG. 22, guidewire acceptor (560) is in communication with guidewire lumen (520) such that guidewire (130) may be inserted into guidewire acceptor (560) all the way through open distal end (556). Frictional forces as previously described for hand-held suction instrument (100) may be provided via shaft assembly (550) in order to increase the required force to move guidewire (130) relative to hand-held suction instrument (500).

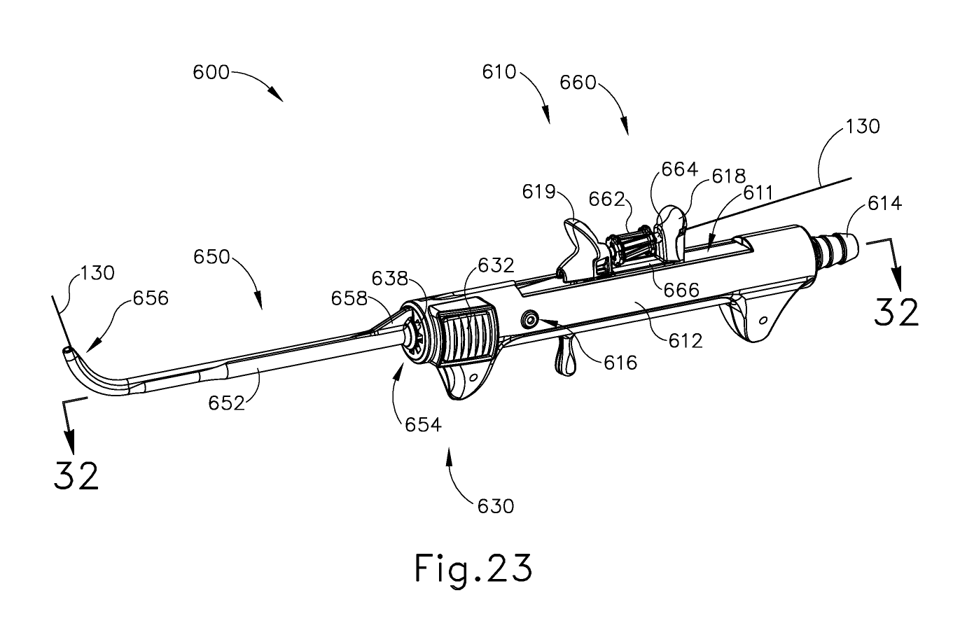

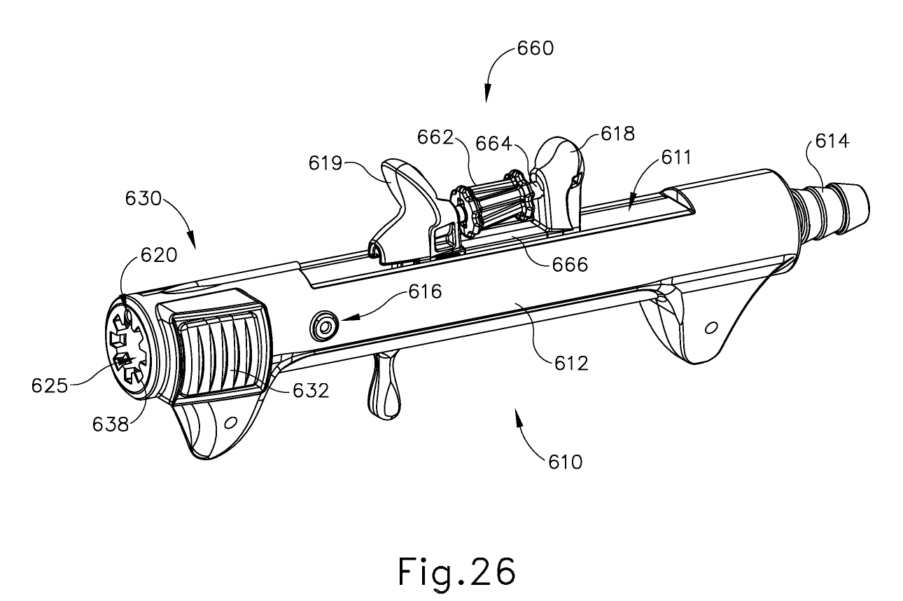

FIGS. 23-32C show another alternative hand-held suction instrument (600) that may be used in place of hand-held suction instrument (100) described above. As shown in FIGS. 23-25, hand-held suction instrument (600) includes a handle assembly (610), a removable shaft assembly (650) extending distally from handle assembly (610), a guidewire manipulation assembly (660) slidably connected to handle assembly (110), and a guidewire (130) extending through a guidewire manipulation assembly (660), shaft assembly (650), and a portion of handle assembly (610). It should be understood that guidewire (130) is substantially similar to guidewire (30) described above. Therefore, a distal end of guidewire (130) may be placed within a patient and located utilizing IGS navigation system (1). Additionally, a proximal end of guidewire (130) may be connected to coupling unit (32) such that guidewire (130) may communication with console (16).

As will also be described below, removable shaft assembly (650) is configured to selectively couple with handle assembly (610); while guidewire (130) is configured to slide within a guidewire lumen (620).

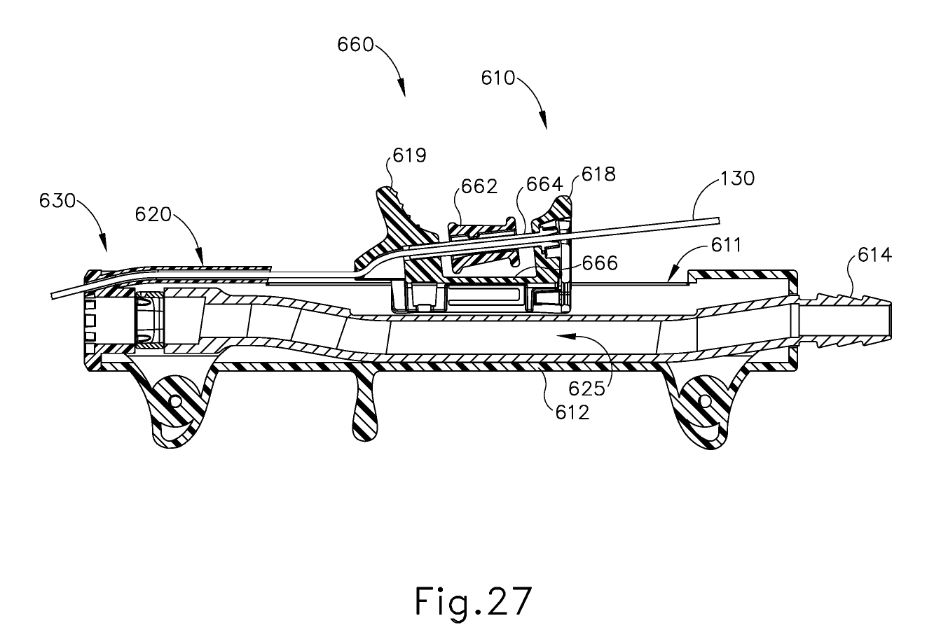

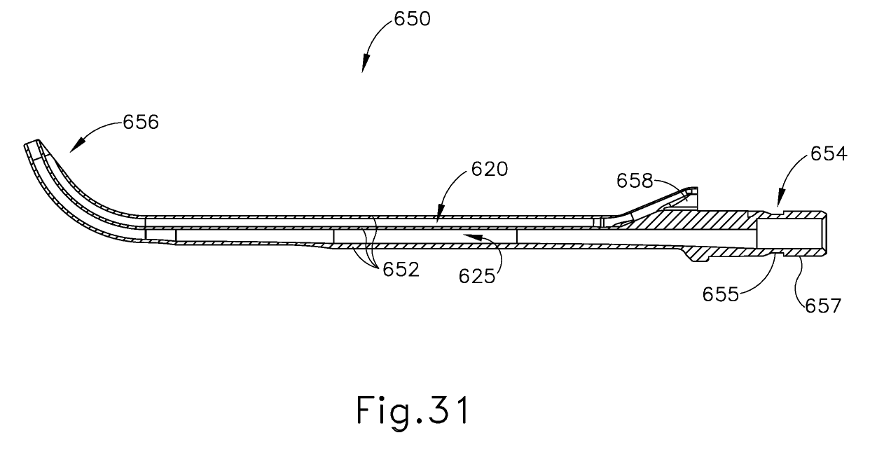

As best seen in FIGS. 27-28 and FIG. 31, removable shaft assembly (650) and handle assembly (610) define guidewire lumen (620) and suction lumen (625), both of which terminate at an open distal end (656) of removable shaft assembly (650). It should be understood that FIG. 31 omits guidewire (130) for purposes of clarity. Similar to guidewire lumen (120), guidewire lumen (620) may slidably and rotatably house a portion of guidewire (130) such that guidewire (130) may rotate about its own longitudinal axis and translate through open distal end (656) of removable shaft assembly (650). Suction lumen (625) extends from a barbed connection (614) through handle assembly (610) and elongate shaft assembly (650), when properly coupled, to provide fluid communication between barbed connection (614) and open distal end (656). It should be understood that portions of handle assembly (610) and shaft assembly (650) that are coupled together in order to define suction lumen (625) form a fluid tight seal. Open distal end (656) and a portion of shaft assembly (650) are dimensioned to be inserted into an anatomical passageway of a patient via a nostril. However, it should be understood open distal end (656) of shaft assembly (650), and the rest of shaft assembly (650), may be dimensioned to be inserted in a variety of anatomical passageways as would be apparent to one having ordinary skill in the art in view of the teachings herein.

Handle assembly (610) includes body (612) defining slot (611) and a suction control port (616), proximal barbed connector (614), and a distal coupling portion (630). Barbed connector (614) is dimensioned to couple with connecting tube (54) in order to provide fluid communication between suction lumen (625) and connecting tube (54). While in the current example barbed connector (614) is utilized to provide fluid communication between connecting tube (54) and suction lumen (625) of hand-held suction instrument (600), any other suitable connector may be utilized as would be apparent to one having ordinary skill in the art in view of the teachings herein.

Body (612) of handle assembly (610) is dimensioned so that an operator may grip handle assembly (610) and control guidewire manipulation assembly (660) with one hand. This may allow for an operator to control hand-held suction instrument (600) with one hand while controlling an endoscope or other instrument with another hand. Therefore, an operator may be able to utilize hand-held suction instrument (600) in conjunction with visuals provided by both IGS navigation system (1) and an endoscope. Alternatively, an operator may utilize hand-held suction instrument (600) with just an endoscope or just IGS navigation system (1).

Suction port (616) is placed on handle such that an operator may selectively cover suction port (616) with the same hand grasping body (612) during use of hand-held suction instrument (600) when properly assembled. Suction port (616) connects the exterior of body (612) with suction lumen (625). Suction port (616) is dimensioned to act as a vent for suction lumen (625) when vacuum assembly (50) is connected and providing suction through suction lumen (625). Therefore, when suction source assembly (52) is activated and providing suction through connecting tube (54) and suction lumen (625), an operator may prevent suction from occurring at open distal end (656) of shaft assembly (650) by leaving suction port (616) open. In other words, suction source assembly (52) will simply draw in atmospheric air via suction port (616) when suction port (616) is open. If an operator desires to provide suction at open distal end (656) of shaft assembly (650), an operator may cover suction port (616). In other words, suction port (616) allows an operator to selectively activate suction at open distal end (656) of shaft assembly (650) even if vacuum assembly (50) is continuously activated and properly connected to hand-held suction instrument (600).

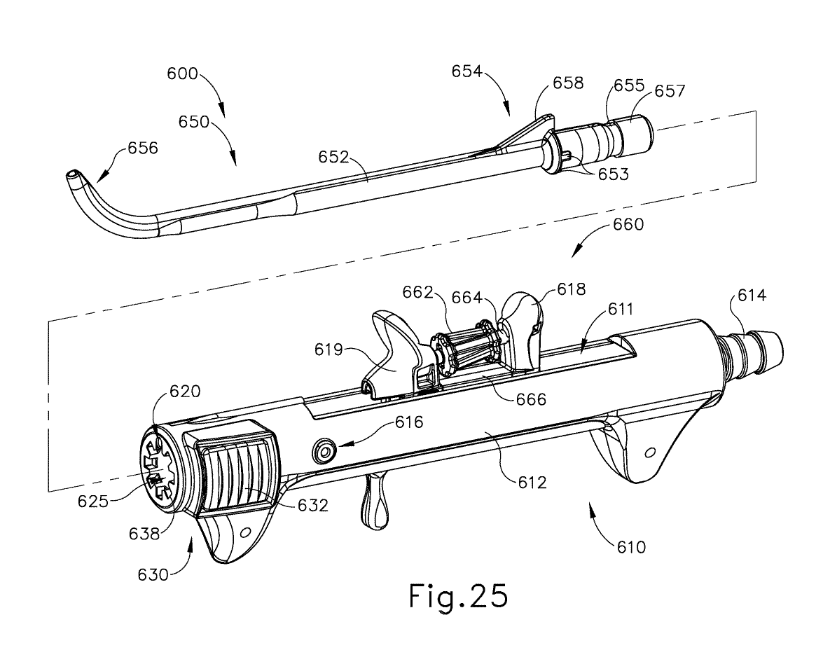

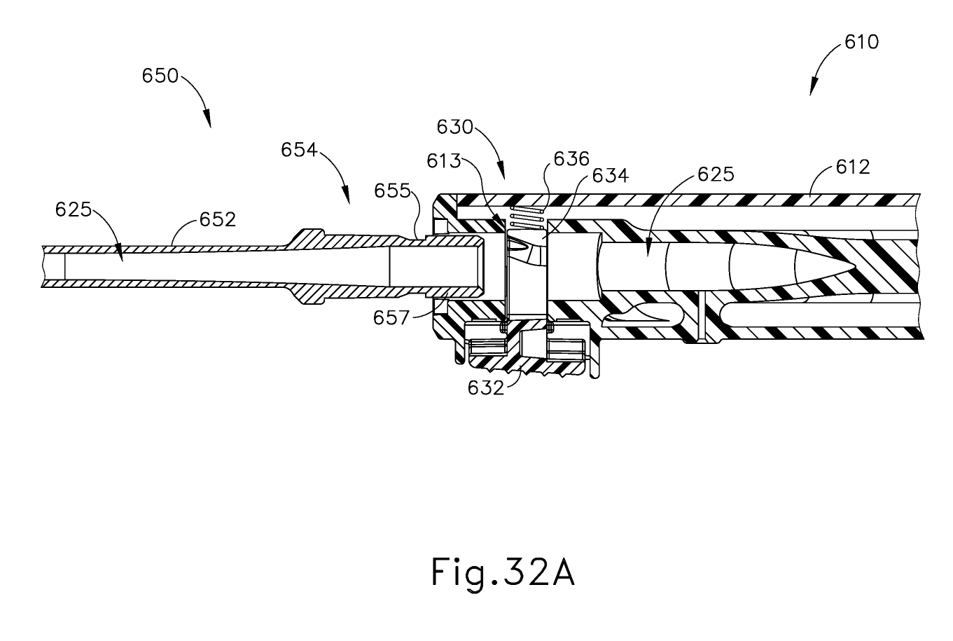

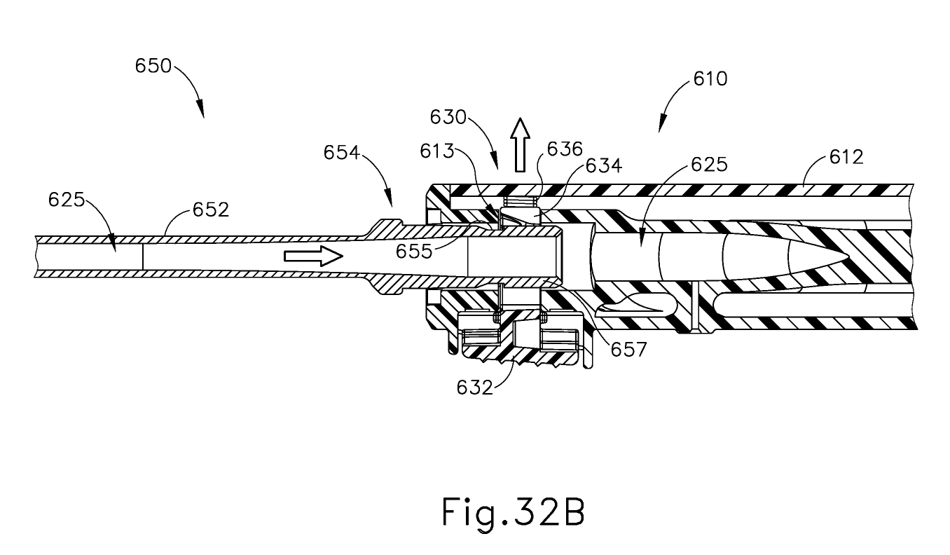

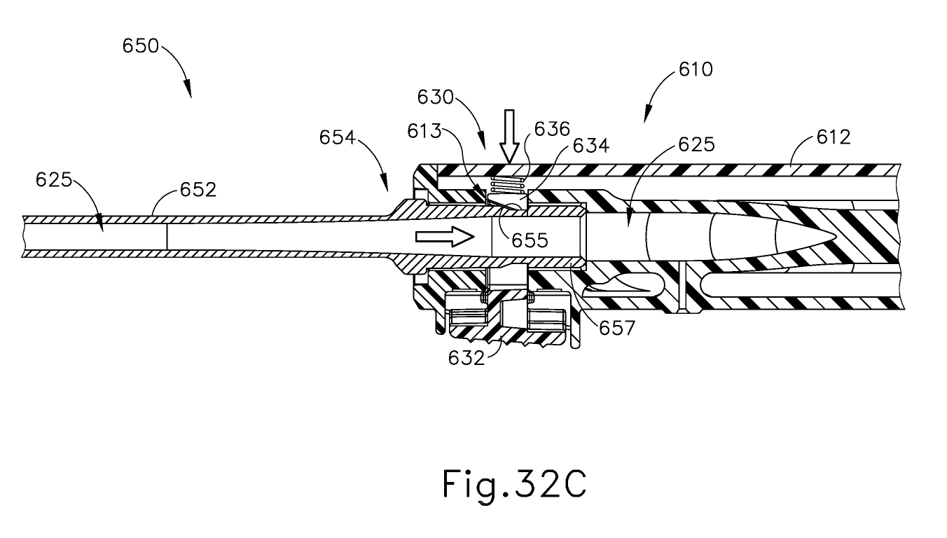

As best seen in FIGS. 25 and 32A-32C, distal coupling portion (630) includes a locking mechanism (632), a latch (634) fixed to locking mechanism (632), a resilient biasing member (636), and an angularly spaced array of radially extending rotational alignment notches (638). A portion of locking mechanism (632) is accessible from an exterior of body (612); while another portion of locking mechanism (632) and latch (634) are housed within a channel (613) defined by body (612). Resilient biasing member (636) is coupled to both body (612) and latch (634) in order to bias latch (634) and locking mechanism (632) to a first position. As will be described in greater detail below, resilient biasing member (636) is configured to allow locking mechanism (632) and latch (634) to selectively couple handle assembly (610) with shaft assembly (650) such that shaft assembly (650) is longitudinally fixed relative to handle assembly (610).

As will be described in greater detail below, slot (611) slidably houses guidewire manipulation assembly (660) such that guidewire manipulation assembly (660) may translate guidewire (130) within guidewire lumen (620) defined by handle assembly (610) and removable shaft assembly (650) when properly coupled.

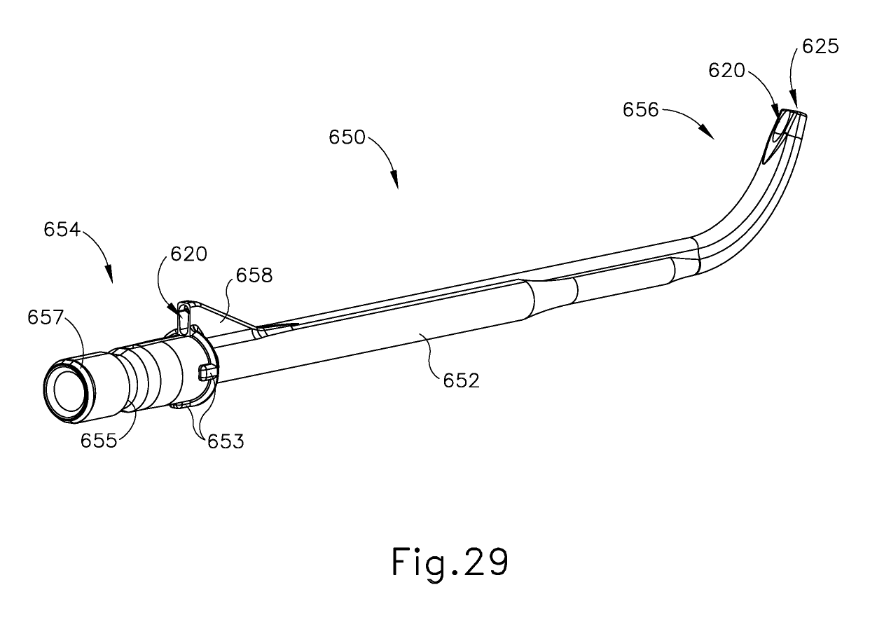

As best shown in FIGS. 29-31, shaft assembly (650) includes a shaft body (652) extending from a proximal coupling portion (654) to open distal end (656). As described above, shaft body (652) defines portions of guidewire lumen (620) and suction lumen (625) such that guidewire (130) may translate through open distal end (656) and suction lumen (625) may carry unwanted fluid/matter away from open distal end (656) when shaft assembly (650) is properly coupled to handle assembly (610). Proximal coupling portion (654) includes a lumen alignment member (658), a plurality of locking protrusions (653), a camming surface (657), and a locking notch (655). Guidewire lumen (620) extends within shaft assembly (650) from lumen alignment member (658) to open distal end (656).

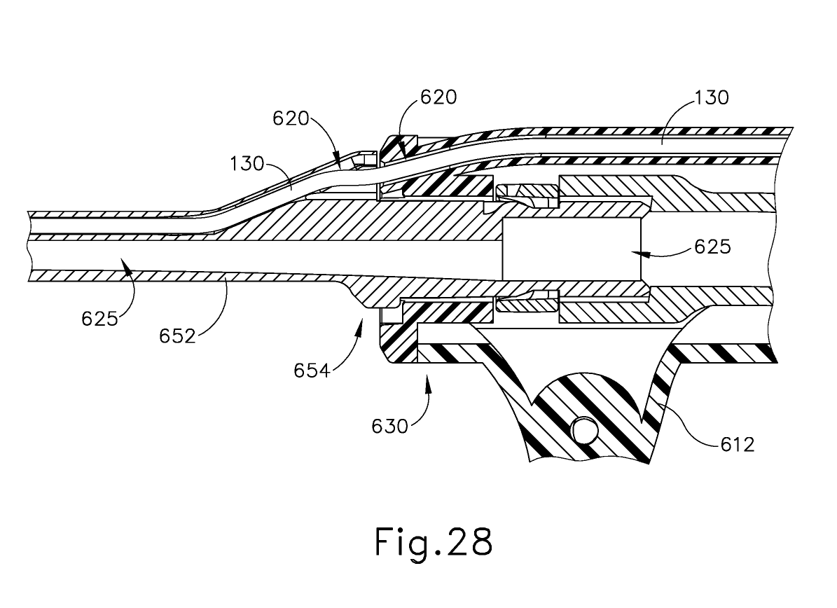

As best seen in FIG. 28, lumen alignment member (658) is configured to align with the terminating end of guidewire lumen (620) defined by distal coupling portion (630) of handle assembly (610). Lumen alignment member (658) is therefore sized to receive guidewire (130) exiting distal coupling portion (630) of handle assembly (610). Locking protrusions (653) are configured to be inserted within rotational alignment notches (638) of distal coupling portion (630) when shaft assembly (650) is properly coupled with handle assembly (610). Locking protrusions (653) and rotation alignment notches (638) prevent replaceable shaft assembly (650) from rotating about its own longitudinal axis relative to handle assembly (610), thereby helping ensure proper alignment of the portions of guidewire lumen (620) transitioning from handle assembly (610) to shaft assembly (650). As will be described in greater detail below, camming surface (657) and locking notch (655) are configured to interact with locking mechanism (632) and latch (634) to selectively couple handle assembly (610) with shaft assembly (650) such that shaft assembly (650) is longitudinally fixed relative to handle assembly (610).

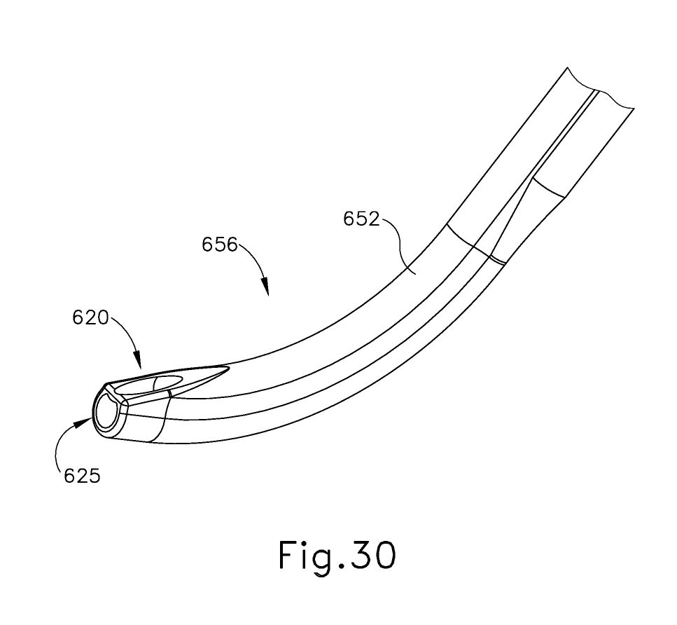

As best seen in FIG. 30, open distal end is bent relative to the longitudinal axis defined by the rest of replaceable shaft assembly (650), and may form any suitable number of angles with the longitudinal axis of handle assembly (610) as would be apparent to one having ordinary skill in the art in view of the teachings herein. Guidewire lumen (620) terminates at a truncated angle at open distal end (656), which may decrease the distal profile of open distal end (656). As best shown in FIG. 31, guidewire lumen (620) and suction lumen (625) may be physically separated by shaft body (652) along the length of shaft assembly (650) to the open distal end (656).

In the current example, proximal coupling portion (654) is parallel with the longitudinal axis defined by shaft body (652), however this is merely optional. Proximal coupling portion (654) may form any suitable number of curves and/or bends relative to the longitudinal axis defined by shaft body (652) as would be apparent to one having ordinary skill in the art in view of the teachings herein.

As best seen shown in FIGS. 23-27, Guidewire manipulation assembly includes a locking grip (662) and a collet (664), which are substantially similar to locking grip (162) and collet (164) described above, with differences elaborated below. Therefore, locking grip (662) is slidably coupled to collet (664), and is configured to translate along collet (664) in order to selectively lock guidewire (130) relative to collet (664). Locking grip (662) is configured to rotate collet (664) and guidewire (130) unitarily together about the longitudinal axis defined by guidewire (130) when guidewire (130) is locked to collet (664).

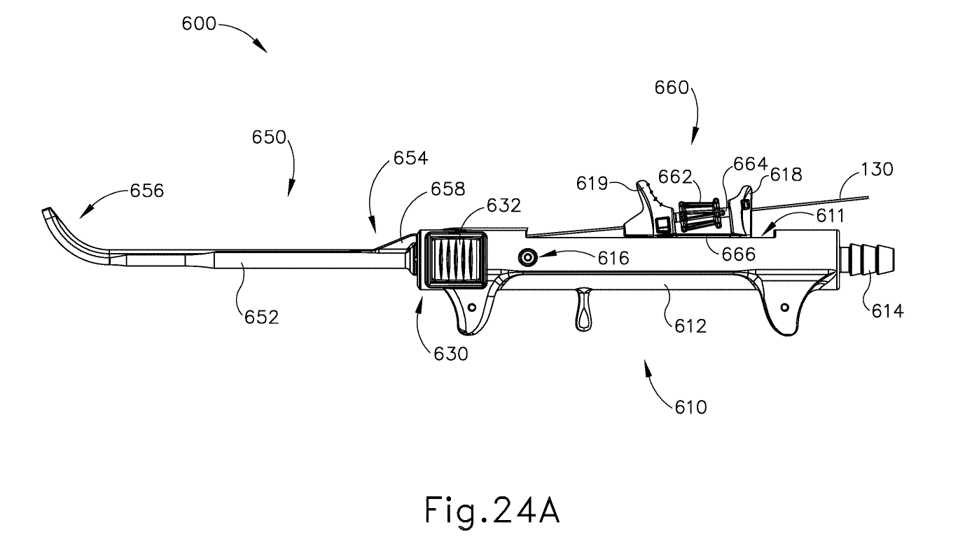

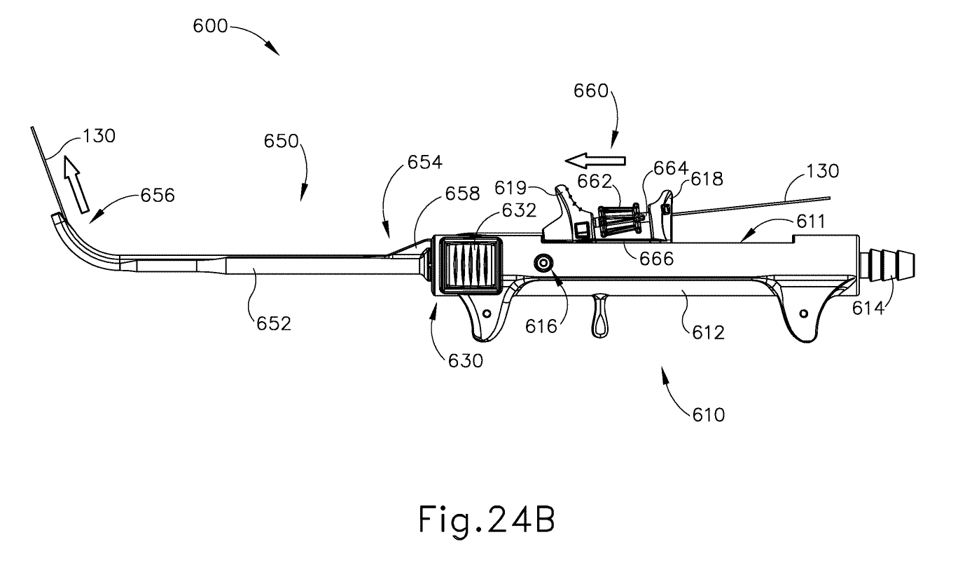

As will be described in greater detail below, guidewire manipulation assembly (660) is also configured to slide within slot (611) defined by body (612) of handle assembly (610) in order to translate guidewire (130) within and through a guidewire lumen (620). Guidewire manipulation assembly (660) also includes a pair of mounts (618, 619) and a sliding base (666). Locking grip (662) and collet (664) are rotatably mounted to mounts (618, 619), similar to how locking grip (162) and collet (164) are rotatably mounted to mounts (118, 119) described above. However, mounts (618, 619) extend vertically from a sliding base (666); while sliding base (666) is configured to slide within slot (611) defined by body (612). Therefore, as shown between FIGS. 24A-24B, an operator may push or pull guidewire manipulation assembly (660) via mounts (618, 619) or locking grip (662) in order to translate guidewire manipulation assembly (660) relative to handle assembly (610). If guidewire (130) is coupled to guidewire manipulation assembly (660), similar to guidewire manipulation assembly (160) described above, guidewire (130) may be translated within guidewire lumen (620) in order to exit open distal end (656) of shaft assembly (650) when properly coupled to handle assembly (610).

As described above, and will be described in greater detail below, shaft assembly (650) may selectively couple with handle assembly (610) via distal coupling portion (630) and proximal coupling portion (654). FIGS. 32A-32C show shaft assembly (650) coupling with handle assembly (650). As shown in FIG. 32A, an operator may insert shaft assembly (650) into handle assembly (650) such that suction lumens (625) defined by both shaft assembly (650) and handle assembly (610) are aligned. It should be understood that lumen alignment member (658) of shaft assembly (650) should also be aligned with the distal termination of guidewire lumen (620) within handle assembly (610). Additionally, it should be understood that locking protrusions (653) are also inserted into corresponding rotational alignment notches (638) such that shaft assembly (650) may not rotate about the longitudinal axis of shaft assembly (650) relative to handle assembly (610).

As shown in FIG. 32B, an operator may further insert shaft assembly (650) into handle assembly (650) such that camming surface (657) pushes against latch (634), thereby pushing latch (634) and resilient biasing member (636) from the first, biased position to a second position closer to body (612) of handle assembly (610).

Next, as shown in FIG. 32C, an operator may further insert shaft assembly (650) into handle assembly (650) such that camming surface (657) no longer pushes against latch (634). Due to the resilient nature of resilient biasing member (636), latch (634) is pushed within locking notch (655) of shaft assembly (650). Therefore, shaft assembly (650) is effectively longitudinally locked relative to handle assembly (610). It should be understood that, at the position shown in FIG. 32C, portions of shat assembly (650) connected with handle assembly (610) forming suction lumen (625) also form a fluid tight seal for suction lumen (625). Forming a fluid tight seal may be accomplished in any suitable number of way known to one having ordinary skill in the art in view of the teachings herein. For example, O-rings may be utilized to form a fluid tight seal for suction lumen (625).

If an operator desires to remove shaft assembly (650) from handle assembly (610), the operator may push the portion of locking mechanism (632) located on the exterior of body (612) inwardly toward suction lumen (625), therefore compressing resilient biasing member (636) and forcing latch (634) towards body (612), which disengages latch (634) from locking notch (655). With latch (634) disengaged with locking notch (655), the operator may pull shaft assembly (650) away from distal coupling portion (630) of handle assembly (610).

III. Additional Devices for Use with Navigation Guidewire

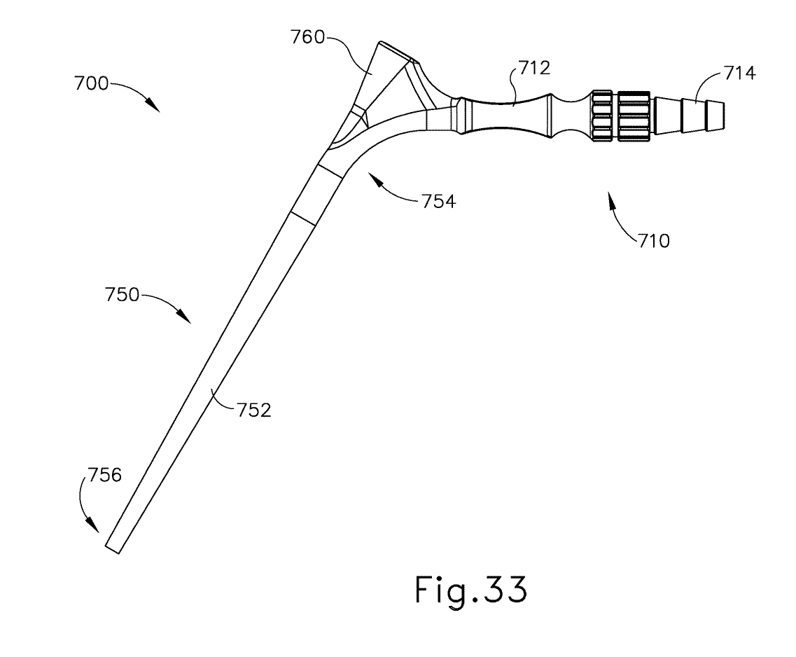

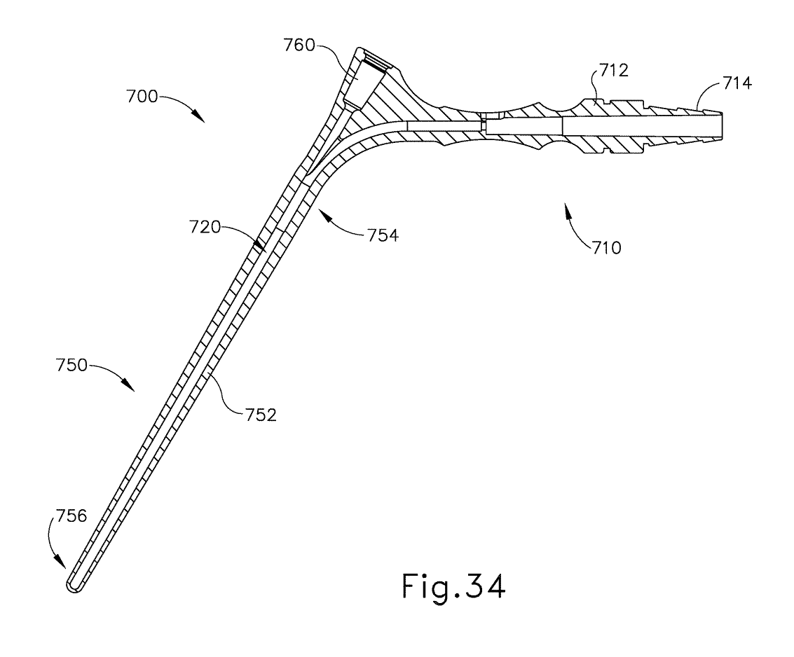

FIGS. 33-34 show an exemplary pointer (700) that may be used in conjunction with guidewire (130) and IGS navigation system (1). Pointer (700) of this example includes a handle assembly (710), a shaft assembly (750), and a guidewire acceptor (760). Handle assembly (710) includes a body (712) and a barbed connection (714). Barbed connection (714) may be dimensioned to mate with any suitable surgical equipment as would be apparent to one having ordinary skill in the art. While in the current example, a barbed connection (714) is utilized, in some other versions no connection may be used, or any other suitable connection may be used as would be apparent to one having ordinary skill in the art in view of the teachings herein.

Body (712) of handle assembly (710) is dimensioned so that an operator may grip handle assembly (710) with one hand. This may allow for an operator to control pointer (700) with one hand while controlling an endoscope or other instrument with another hand. Therefore, an operator may be able to utilize pointer (700) in conjunction with visuals provided by both IGS navigation system (1) and an endoscope. Alternatively, an operator may utilize pointer (710) with just an endoscope or just IGS navigation system (1).

Shaft assembly (750) includes a shaft body (752) extending from a bent proximal portion (754) to distal end (756). Shaft body (752) defines guidewire lumen (720) such that guidewire (130) may translate to distal end (756). Therefore, guidewire (130) may be inserted into guidewire lumen (720) such that IGS navigation system (1) may determine the location of distal end 9756) of pointer (700). Bent proximal portion (154) bends the rest of elongate shaft assembly (750) away from the longitudinal axis of handle assembly (710), such that shaft body (752) defines an oblique angle with the longitudinal axis of handle assembly (710). Bent proximal portion (754) may form any suitable number of angles with the longitudinal axis of handle assembly (710) as would be apparent to one having ordinary skill in the art in view of the teachings herein. In the current example, distal end (756) is parallel with the longitudinal axis defined by shaft body (752), however this is merely optional. In the present example, distal end (756) is closed, such that a guidewire disposed in guidewire lumen (720) will not exit distal end (756) distally. Distal end (756) may form any suitable number of curves and/or bends relative to the longitudinal axis defined by shaft body (752) as would be apparent to one having ordinary skill in the art in view of the teachings herein.

Guidewire acceptor (760) is dimensioned to receive guidewire (130) such that guidewire (130) may be inserted in guidewire lumen (720) toward distal end (756).

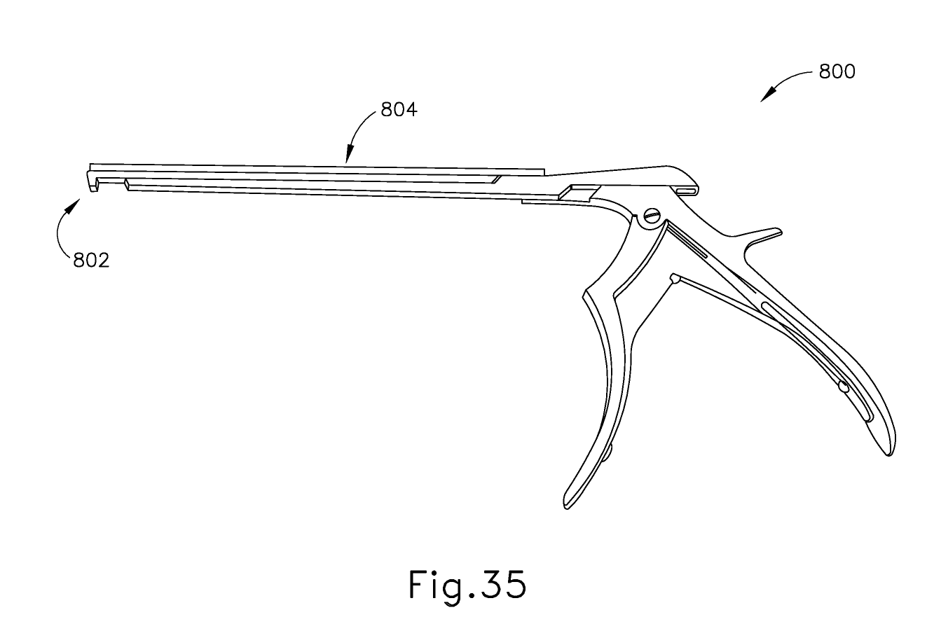

FIG. 35 shows a Kerrison instrument (800) having a distal end (802) and a guidewire lumen (804) extending toward distal end (802). Kerrison instrument (800) of the present example is configured and operable just like a conventional Kerrison instrument, except that this example of Kerrison instrument (800) includes guidewire lumen (804). Guidewire lumen (804) is dimensioned to receive guidewire (130) such that distal end of guidewire (130) may be located adjacent to distal end (802) of Kerrison instrument (800). Therefore, IGS navigation system (1) may be utilized to determine the location of distal end of guidewire (130), which may be associated with distal end (802) of Kerrison instrument (804). In other words, guidewire lumen (804) provides IGS navigational capabilities to Kerrison instrument (800) by accepting guidewire (130).

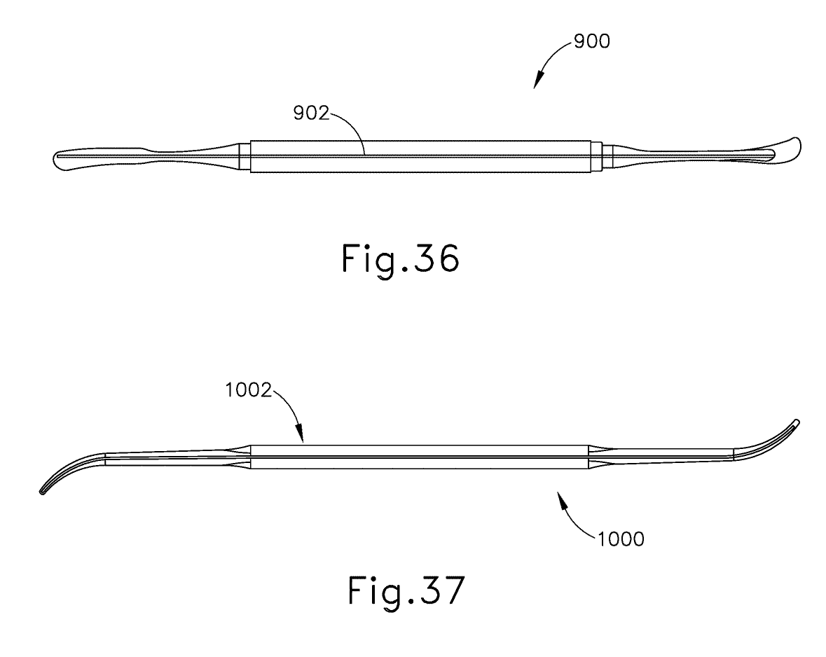

FIG. 36 shows a Freer elevator (900) having a separate lumen (902) following the profile of Freer elevator (900). Freer elevator (900) of the present example is configured and operable just like a conventional Freer elevator instrument, except that this example of Freer elevator (900) includes lumen (902). Lumen (902) is configured to receive guidewire (130). Therefore, IGS navigation system (1) may be utilized to determine the location of the distal end of guidewire (130), which may be associated with a desired location along Freer elevator (900). In other words, lumen (902) provides IGS navigational capabilities to Freer elevator (900) by accepting guidewire (130).

FIG. 37 shows a sinus seeker (1000) having a separate lumen (1002) following the profile of sinus seeker (1000). Sinus seeker (1000) of the present example is configured and operable just like a conventional seeker instrument, except that this example of sinus seeker (1000) includes lumen (1002). Lumen (1002) is configured to receive guidewire (130). Therefore, IGS navigation system (1) may be utilized to determine the location of the distal end of guidewire (130), which may be associated with a desired location along sinus seeker (1000). In other words, lumen (1002) provides IGS navigational capabilities to sinus seeker (1000) by accepting guidewire (130).

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) a navigational guidewire, wherein the navigational guidewire comprises: (i) a sensing element, wherein the sensing element is configured to respond to positioning within an electromagnetic field, (ii) an outer member, and (iii) a conductor extending along the length of the outer member, wherein the conductor is in communication with the sensing element; and (b) a suction device comprising a shaft assembly, wherein the shaft assembly comprises an open distal end, wherein the shaft assembly defines a suction lumen and a guidewire lumen extending through the open distal end, wherein the guidewire lumen is dimensioned to receive the sensing element of the navigational guidewire.

### Example 2

The apparatus of Example 1, further comprising a handle assembly connected to the shaft assembly.

### Example 3

The apparatus of Example 2, wherein the suction lumen extends through the handle assembly.

### Example 4

The apparatus of Example 3, wherein the handle assembly further comprises a connector configured to couple the suction lumen with a suction source.

### Example 5

The apparatus of Example 4, wherein the handle assembly further defines a suction control port configured to selectively divert suction from a portion of the suction lumen within the handle to a portion of the section lumen adjacent to the open distal end.

### Example 6

The apparatus of any one or more of Examples 2 through 5, wherein the shaft assembly further comprises a bent proximal portion, wherein the bent proximal portion is connected to the handle assembly.

### Example 7

The apparatus of any one or more of Examples 1 through 6, wherein the shaft assembly further comprises a bent proximal portion, wherein the bent proximal portion is connected to the handle assembly.

### Example 8

The apparatus of any one or more of Examples 1 through 7, wherein the guidewire lumen is saddled within the suction lumen.

### Example 9

The apparatus of any one or more of Examples 1 through 8, further comprising a guidewire manipulation assembly configured to receive the navigational guidewire.

### Example 10

The apparatus of Example 9, wherein the guidewire manipulation assembly is configured to transition between an unlocked configuration and a locked configuration, wherein the navigational guidewire is fixed relative to the guidewire manipulation assembly in the locked configuration.

### Example 11

The apparatus of Example 10, wherein the guidewire manipulation assembly is configured to rotate the navigational guidewire about a longitudinal axis defined by the navigational guidewire.

### Example 12

The apparatus of Example 11, wherein the guidewire manipulation assembly comprises a collet and a locking grip, wherein the locking grip is configured to slide along the collet to transition the guidewire manipulation assembly between the unlocked configuration and the locked configuration.

### Example 13

The apparatus of any one or more of Examples 11 through 12, wherein the guidewire manipulation assembly is configured to translate relative to the handle assembly.

### Example 14

The apparatus of any one or more of Examples 1 through 13, wherein the shaft assembly comprises a body, wherein the body partially defines the guidewire lumen having a “U” shape.

### Example 15

The apparatus of any one or more of Examples 11 through 14, further comprising a heat shrink surrounding the body of the shaft assembly.

### Example 16

The apparatus of any one or more of Examples 1 through 15, wherein the open distal end comprises a ball tip.

### Example 17

The apparatus of any one or more of Examples 2 through 17, wherein the shaft assembly if detachable from the handle assembly.

### Example 18

The apparatus of Example 17, wherein the handle assembly comprises coupling assembly configured to selectively couple with the shaft assembly.

### Example 19

An apparatus comprising: (a) a navigational guidewire, wherein the navigational guidewire comprises: (i) a sensing element, wherein the sensing element is configured to respond to positioning within an electromagnetic field, (ii) an outer member, and (iii) a conductor extending along the length of the outer member, wherein the conductor is in communication with the sensing element; and (b) a suction device comprising: (i) a handle assembly, wherein the handle assembly defines a longitudinal axis, wherein the handle assembly further comprises a suction port, and (ii) a shaft assembly, wherein the shaft assembly extends obliquely from the handle assembly, wherein the shaft assembly comprises an open distal end, wherein the shaft assembly defines a suction lumen and a guidewire lumen extending through the open distal end, wherein the suction lumen is in fluid communication with the suction port, wherein the guidewire lumen is dimensioned to receive the sensing element of the navigational guidewire

### Example 20

An apparatus comprising: (a) a navigational guidewire, wherein the navigational guidewire comprises: (i) a sensing element, wherein the sensing element is configured to respond to positioning within an electromagnetic field, (ii) an outer member, and (iii) a conductor extending along the length of the outer member, wherein the conductor is in communication with the sensing element; and (b) an instrument defining a lumen, wherein the lumen is configured to receive the navigational guidewire, wherein the instrument is selected from the group consisting of a Kerrison instrument, a Freer elevator, and a sinus seeker

V. Miscellaneous

Various examples herein provide a guidewire lumen (120, 220, 320, 420, 520, 620) through which a guidewire may be disposed adjacent to a corresponding suction lumen (125, 225, 325, 425, 525, 625). If should be understood that such examples may also permit various other kinds of instruments and components to be disposed in guidewire lumen (120, 220, 320, 420, 520, 620). In other words, the utility of guidewire lumen (120, 220, 320, 420, 520, 620) need not be limited solely to receipt of a guidewire. By way of example only, some versions of the devices described herein may permit a laser source, forceps, dilation balloon, elevator, interventional catheter, and/or other instrument or component to be passed through (or otherwise disposed in) guidewire lumen (120, 220, 320, 420, 520, 620). Various suitable kinds of instruments or components that may be passed through (or otherwise disposed in) guidewire lumen (120, 220, 320, 420, 520, 620) will be apparent to those of ordinary skill in the art in view of the teachings herein.

In the present example, shaft body (152, 252, 352, 452, 652, 754) is substantially rigid. Thus, the orientation and configuration of shaft body (152, 252, 352, 452, 652, 754) relative to handle assembly (112, 510, 610) is predetermined and unchangeable. In some other versions, at least a portion of shaft body (152, 252, 352, 452, 652, 754) is malleable or otherwise bendable. In such versions, the operator may manipulate shaft body (152, 252, 352, 452, 652, 754) to thereby provide a different orientation and/or configuration of shaft body (152, 252, 352, 452, 652, 754) relative to handle assembly (112, 510, 610). This capability may facilitate usage of the instrument in various anatomical regions.

In some versions, shaft body (152, 252, 352, 452, 652, 754) has a substantially circular cross-sectional profile. In some other versions, shaft body (152, 252, 352, 452, 652, 754) has an elliptical cross-sectional profile. An elliptical cross-sectional profile may provide better access for other instruments adjacent to shaft body (152, 252, 352, 452, 652, 754) within a nasal cavity, such that shaft body (152, 252, 352, 452, 652, 754) and another instrument may be readily inserted in the same nasal cavity at the same time. Other suitable cross-sectional profiles that may be incorporated into shaft body (152, 252, 352, 452, 652, 754) will be apparent to those of ordinary skill in the art in view of the teachings herein.

It should also be understood that the various devices herein may include a marking or other indicia on a handheld portion of the device to indicate to the operator the positioning of guidewire lumen (120, 220, 320, 420, 520, 620) and/or suction lumen (125, 225, 325, 425, 525, 625). For instance, handle assembly (112, 510, 610) may include a first marking indicating the angular position of guidewire lumen (120, 220, 320, 420, 520, 620) and/or a second marking indicating the angular position of suction lumen (125, 225, 325, 425, 525, 625). The operator may find it beneficial of such markings provide the operator with a better understanding of the location of suction lumen (125, 225, 325, 425, 525, 625) in relation to guidewire lumen (120, 220, 320, 420, 520, 620) (i.e., the angular position of suction lumen (125, 225, 325, 425, 525, 625) about the longitudinal axis defined by guidewire lumen (120, 220, 320, 420, 520, 620)). In particular, once the operator knows the location of the distal end of guidewire lumen (120, 220, 320, 420, 520, 620) within the patient, the operator may observe the indicia on handle assembly (112, 510, 610) (or on some other component that is external to the patient) to identify the precise location of the distal end of suction lumen (125, 225, 325, 425, 525, 625) within the patient. Various suitable forms that such indicia may take, and various suitable positions at which such indicia may be located, will be apparent to those of ordinary skill in the art in view of the teachings herein.

The examples described above include the incorporation of a navigation coil or other navigation sensor in the distal end of guidewire (30, 130) to enable navigation and guidance via IGS system (1). In addition to, or in lieu of, providing such a navigation coil or other navigation sensor in the distal end of guidewire (30, 130), some versions may also incorporate one or more navigation coils or other navigation sensors in one or more other locations. By way of example only, one or more other navigation coils or other navigation sensors in handle assembly (112, 510, 610) and/or in some other component that will remain external to the patient during use of the device. Various other suitable locations where one or more other navigation coils or other navigation sensors may be provided will be apparent to those of ordinary skill in the art in view of the teachings herein.

Any of the devices herein may be modified and/or used in accordance with at least some of the teachings of U.S. Pub. No. 2016/0310042, entitled “System and Method to Map Structures of Nasal Cavity,” published Oct. 27, 2016, the disclosure of which is incorporated by reference herein. Thus, the devices herein may be used to provide mapping of anatomy within and adjacent to a patient's nasal cavity. Similarly, the devices herein may be used to provide probing of anatomy within and adjacent to a patient's nasal cavity.

Any of the devices herein may be formed using 3D printing and/or using any other suitable manufacturing technique(s).

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. In some instances, the instrument may be placed in a reprocessing tray (e.g., a metal bin or basket) and then cleaned in a surgical instrument washer. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, steam, hydrogen peroxide vapor (e.g., via a STERRAD sterilization system by Advanced Sterilization Products of Irvine, Calif.), and/or using any other suitable systems or techniques.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) a navigational guidewire, wherein the navigational guidewire comprises:
(i) a sensing element, wherein the sensing element is configured to respond to positioning within an electromagnetic field,
(ii) an outer member, and
(iii) a conductor extending along the length of the outer member, wherein the conductor is in communication with the sensing element; and
(b) a suction device comprising a shaft assembly, wherein the shaft assembly comprises an open distal end, wherein the shaft assembly defines a suction lumen and a guidewire lumen extending through the open distal end, wherein the guidewire lumen is dimensioned to receive the sensing element of the navigational guidewire.

2. The apparatus of claim 1, further comprising a handle assembly connected to the shaft assembly.

3. The apparatus of claim 2, wherein the suction lumen extends through the handle assembly.

4. The apparatus of claim 3, wherein the handle assembly further comprises a connector configured to couple the suction lumen with a suction source.

5. The apparatus of claim 4, wherein the handle assembly further defines a suction control port configured to selectively divert suction from a portion of the suction lumen within the handle to a portion of the section lumen adjacent to the open distal end.

6. The apparatus of claim 2, wherein the shaft assembly further comprises a bent proximal portion, wherein the bent proximal portion is connected to the handle assembly.

7. The apparatus of claim 1, wherein a portion of the suction lumen and the guidewire lumen are in fluid communication.

8. The apparatus of claim 1, wherein the guidewire lumen is saddled within the suction lumen.

9. The apparatus of claim 1, further comprising a guidewire manipulation assembly configured to receive the navigational guidewire.

10. The apparatus of claim 9, wherein the guidewire manipulation assembly is configured to transition between an unlocked configuration and a locked configuration, wherein the navigational guidewire is fixed relative to the guidewire manipulation assembly in the locked configuration.

11. The apparatus of claim 10, wherein the guidewire manipulation assembly is configured to rotate the navigational guidewire about a longitudinal axis defined by the navigational guidewire.

12. The apparatus of claim 11, wherein the guidewire manipulation assembly comprises a collet and a locking grip, wherein the locking grip is configured to slide along the collet to transition the guidewire manipulation assembly between the unlocked configuration and the locked configuration.

13. The apparatus of claim 11, wherein the guidewire manipulation assembly is configured to translate relative to the handle assembly.

14. The apparatus of claim 1, wherein the shaft assembly comprises a body, wherein the body partially defines the guidewire lumen having a “U” shape.

15. The apparatus of claim 12, further comprising a heat shrink surrounding the body of the shaft assembly.

16. The apparatus of claim 1, wherein the open distal end comprises a ball tip.

17. The apparatus of claim 2, wherein the shaft assembly if detachable from the handle assembly.

18. The apparatus of claim 17, wherein the handle assembly comprises coupling assembly configured to selectively couple with the shaft assembly.

19. An apparatus comprising:
(a) a navigational guidewire, wherein the navigational guidewire comprises:
(i) a sensing element, wherein the sensing element is configured to respond to positioning within an electromagnetic field,
(ii) an outer member, and
(iii) a conductor extending along the length of the outer member, wherein the conductor is in communication with the sensing element; and
(b) a suction device comprising:
(i) a handle assembly, wherein the handle assembly defines a longitudinal axis, wherein the handle assembly further comprises a suction port, and
(ii) a shaft assembly, wherein the shaft assembly extends obliquely from the handle assembly, wherein the shaft assembly comprises an open distal end, wherein the shaft assembly defines a suction lumen and a guidewire lumen extending through the open distal end, wherein the suction lumen is in fluid communication with the suction port, wherein the guidewire lumen is dimensioned to receive the sensing element of the navigational guidewire.

20. An apparatus comprising:
(a) a navigational guidewire, wherein the navigational guidewire comprises:
(i) a sensing element, wherein the sensing element is configured to respond to positioning within an electromagnetic field,
(ii) an outer member, and
(iii) a conductor extending along the length of the outer member, wherein the conductor is in communication with the sensing element; and
(b) an instrument defining a lumen, wherein the lumen is configured to receive the navigational guidewire, wherein the instrument is selected from the group consisting of a Kerrison instrument, a Freer elevator, and a sinus seeker.
